(19) United States
(12) Patent Application Publication
(10) Pub. No.: US 2023/0126423 A1
(43) Pub. Date: Apr. 27, 2023

(54) ENHANCED EXPRESSION YIELD OF IMMUNOGLOBULIN A IN EUKARYOTES

(71) Applicant: Ventria Bioscience Inc., Junction City, KS (US)

(72) Inventors: Deshui Zhang, Junction City, KS (US); Andrew Simon, Junction City, KS (US); Derek Schneweis, Junction City, KS (US); Saurav Misra, Junction City, KS (US); Javier Herrera, Junction City, KS (US); Mark Lagrimini, Junction City, KS (US)

(21) Appl. No.: 17/908,640

(22) PCT Filed: Mar. 2, 2021
(86) PCT No.: PCT/US21/20437

§ 371 Date: Sep. 1, 2022

Related U.S. Application Data
(60) Provisional application No. 62/984,162, filed on Mar. 2, 2020.

(51) Int. Cl. C12N 15/82; C12N 15/62; C07K 16/00

(52) U.S. Cl. CPC C12N 15/8258; C12N 15/625; C07K 16/00; A61K 2039/505

(57) ABSTRACT

Methods of producing recombinant, multi-component proteins in eukaryotic expression systems, comprising co-transforming a eukaryotic cell with two or more different nucleic acid constructs, each comprising a respective transcriptional unit encoding a protein component, wherein each nucleic acid construct comprises the same promoter and signal sequence, such that each of the components will be targeted to the same organelle of the cell for expression and intracellular assembly. In one or more embodiments, each nucleic acid construct comprises a promoter from a protein storage gene that is operably linked to a DNA sequence that encodes for a protein storage-specific signal sequence.

### CROSS-REFERENCE TO RELATED APPLICATIONS

The present application claims the priority benefit of U.S. Provisional Patent Application Ser. No. 62/984,162, filed Mar. 2, 2020, entitled ENHANCED EXPRESSION YIELD OF IMMUNOGLOBULIN A IN EUKARYOTES, incorporated by reference in its entirety herein.

### SEQUENCE LISTING

The following application contains a sequence listing in computer readable format (CRF), submitted as a text file in ASCII format entitled “SequenceListing,” created on Mar. 2, 2021, as 46 KB. The content of the CRF is hereby incorporated by reference.

### BACKGROUND OF THE INVENTION

### Field of the Invention

The present invention relates to recombinant proteins and intracellular expression of assembled recombinant proteins, for example, IgA.

### Description of Related Art

Antibodies, also called immunoglobulins (Igs), are one of the most active categories of biomolecules currently in medical use and in clinical and preclinical development. Antibodies are critical mediators of the humoral immune response. Immunoglobulins bind and neutralize pathogens and foreign antigens, such as bacteria, fungi, parasites, viruses, and toxins. Moreover, antibody-bound pathogens are detected by receptors on specific immune cells that can engulf or destroy the pathogen. The five major classes or isotypes of antibodies—immunoglobulin G (IgG), immunoglobulin A (IgA), immunoglobulin D (IgD), immunoglobulin E (IgE), and immunoglobulin M (IgM)—differ in size, structure, tissue/organ distribution and major functional roles.

IgG is the major antibody in the blood and constitutes 75% to 80% of the total antibody in human serum. IgD is mainly found on the membranes of maturing B-lymphocytes, where it functions in the activation of these antibody-manufacturing immune cells. IgE is present in trace amounts in the blood, where it is involved in anti-parasite immunity. The large, pentameric IgM makes up about 8% of the antibody in the blood in its secreted form. Blood IgM acts early in infections to neutralize pathogens and activates the complement cascade to proteolytically destroy antibody-bound pathogens or antigens. Finally, IgA is the second most abundant type of antibody in human serum, constituting about 13% of all serum antibodies. However, IgA is the most abundant antibody in the mucosa, including the digestive, respiratory, and reproductive tracts. IgA is also abundant in extravascular secretions including saliva, tears, sweat, milk, and colostrum (early mammary gland secretions that precede “normal” mother's milk, but are a rich source of immune protection for newborns). As such, IgA is the most abundant antibody class in the human body, outnumbering all other antibody isotypes combined. IgA is also the most abundant antibody in mucus, and it forms part of the first line of defense against infectious agents.

The ˜150 kilodalton (kDa) forms of all antibody isotypes, including IgA, are composed of four polypeptide chains: two copies of a ˜25 kDa light chain (LC) and two copies of a ˜50 kDa heavy chain (HC). Each LC associates with an HC to form a heterodimer that is covalently linked by disulfide bonds. In turn, the HCs homodimerize so that the individual heterodimers assemble into a characteristic Y-shaped heterotetrameric structure (FIG. 1). The LC is composed of an amino-terminal variable (V_{L}) domain and one carboxyl-terminal constant (C_{L}) domain, while the HC is composed of an amino-terminal variable domain (V_{H}) and three constant domains (C_{H}1, C_{H}2 and C_{H}3). The V_{L} and V_{H} domains are subdivided into conserved framework sequences and hypervariable or complementary determining regions (CDR). Located near the tips of the immunoglobulin Y “arms”, the closely apposed V_{L} and V_{H} domains of each LC-HC heterodimer form a specific antigen-binding site, so that each monomeric immunoglobulin contains two binding sites. Taken together, the V_{L} and V_{H} domains together with the C_{L} and C_{H}1 domains are termed the Fab (Fragment antigen binding) region. The “stem” or “tail” region of the Y-shaped antibody, composed of the homodimerized C_{H}2 and C_{H}3 domains of the two HCs, is also called the Fc (fragment crystallizable) region. Among other functions, Fc is essential for activating the immune system by binding to specific immunoglobulin receptors and other effector molecules.

In vivo, IgA exists in a monomeric form (mIgA), containing only heavy chain (HC) and light chain (LC), or as secretory IgA (sIgA). While blood/serum IgA is primarily a single Y-shaped immunoglobulin, most IgA in the mucosa and in secretions such as colostrum is present as secretory IgA. Structurally, sIgA contains two complete IgA molecules. An HC from each IgA is linked to an HC of the other IgA through disulfide bonding with a single 15 kDa polypeptide termed a joining chain (JC or J chain). An 80 kDa polypeptide termed a secretory component (SC) associates with the JC-linked IgA dimer to complete the sIgA (FIG. 1). It has been suggested that the SC helps to confer stability and resistance to proteolysis and/or increased sIgA lifetime at surfaces with high proteolytic activity, such as the gut lumen. sIgA exhibits high avidity for polyvalent antigens and targets, associated with its four antigen-binding sites and its ready ability to form oligomeric networks when bound to larger targets. Correspondingly, sIgA plays a major role in immune protection by neutralizing viruses, inhibiting adherence of bacteria, and preventing colonization and penetration of mucosal surfaces by various pathogens.

Because of their critical roles in defense against various pathogenic organisms and agents, and their very high affinities for their specific antigens, antibodies have been used for diverse applications in research, diagnostics and therapy, and in a diverse array of pathologies, including cancers, immune and inflammatory disorders, and against infectious agents. So far, approved antibodies are exclusively of the IgG class, involving either chimeric or humanized IgGs, or fully human monoclonal IgGs. However, more than 95% of infections and pathogens originate at or come into contact with the mucosal system, where IgA is the major class of antibody. The human mucosal system has an approximately 400 m² surface area and likely represents the largest area of exposure of the body to pathogens. Therefore, IgA antibodies represent a valuable class of therapeutic drug proteins for treatment of a wide range of diseases.

Secretory IgAs (sIgA), which are highly enriched on the mucosal surfaces of the human body, are of particular significance due to their likely robustness in the mucosal environment, potentially high therapeutic efficacies, potentially favorable pharmacological profiles and ability to activate immune response pathways inaccessible to IgGs. As many pathogenic infectious agents and diseases engage the human body at the mucosa, the development of sIgAs as therapeutic agents that can be delivered at suitable doses is an area of great interest. Such efforts, however, are severely hampered by the lack of robust, effective, and scalable sIgA expression systems.

All antibodies, but especially multimeric immunoglobulin assemblies such as sIgA, contain numerous intra- and inter-chain disulfide bonds and post-translational modifications, particularly oligosaccharides (glycosylation). The structural complexity and modifications of antibodies necessitate a sophisticated folding apparatus as well as an oxidizing environment for the generation of disulfide bonds. In cells in which they are natively synthesized, antibodies are co-translationally inserted into the endoplasmic reticulum (ER) where they undergo folding, multichain assembly and N-liked glycosylation; subsequently, antibodies are sorted into secretory pathways to be transported to the plasma membrane or the extracellular medium; en route, the antibodies may undergo additional post-translational modification. Improperly folded or assembled antibodies are retained in the ER or may be returned to the ER from the secretory pathway, followed by disposal through proteolytic degradation. Due to the requirement for a complex processing compartment such as the ER, many commonly used prokaryotic expression hosts, such as E. coli, are not capable of efficient production of antibodies. Smaller engineered antibody fragments, such as single-chain variable fragment (scFv) and fragment antigen-binding (Fab) constructs, may possess the antigen binding capacity of the parent antibody. While such fragments may be produced in simpler expression systems, they lack the Fc regions, which mediate immune effector functions that are critical in many therapeutic applications. Eukaryotic cell systems possess the advanced folding, post-translational, and secretion apparatus that satisfy the requirements of producing complete antibodies, though with widely varying efficiency and yield.

Therapeutic protocols that use antibody drugs require very high doses to achieve optimal clinical efficacy, in the range of hundreds of milligrams per dose. Over 95% of currently approved therapeutic antibodies are produced in mammalian cell lines such as CHO (Chinese Hamster Ovary) cell culture. Large quantities of antibodies, in turn, require large volumes of mammalian cultured cells that express the antibody. The expressed antibodies must also be purified to regulated levels of purity and homogeneity using experimentally sophisticated extraction and purification procedures under Current Good Manufacturing Practice (cGMP) conditions. As a result, very high production costs represent a major drawback of mammalian cell-based antibody production. A second major drawback is that mammalian cell culture, as well as other animal-based expression systems and sources of antibodies, are at relatively high risk of contamination by adventitious pathogens including viruses and prions, which then also contaminate the produced antibodies.

While most of the antibodies that have been approved or under clinical development for therapeutic applications belong to the IgG class, there is increasing interest in exploring SIgA antibodies, especially for therapeutic treatments that directly target mucosal immunity and pathology. Because sIgA is assembled from four rather than only two distinct types of polypeptide chains, and because of its more complex architecture, production of sIgA is much more challenging than production of IgG antibodies. Selected eukaryotic expression systems including plants and mammalian cells have been used to express small quantities of bioactive recombinant sIgA. None of these systems approaches the yields required for commercial therapeutic-scale production of sIgA antibodies.

In vivo, the components of native sIgA are produced by two distinct cell types, plasma immune cells and mucosal epithelial cells. The final assembly of the complete sIgA architecture occurs on the surfaces of epithelia. To mimic native assembly processes, several early sIgA production attempts utilized recombinant sIgA components, with each component being expressed in a different cell type. SIgA production by in vitro reconstitution, involving mixing of purified polymeric IgA (pIgA) with SC was also tested. Production methods that use multiple expression cell lines inevitably increase the manufacturing complexity and cost. Single cell-line based methods to produce sIgA were also evaluated, but could not produce a yield that was practical from either an economic or therapeutic standpoint.

Plant cells are emerging as a promising alternative expression system for production of antibodies and other types of recombinant proteins, especially when large amounts are required for commercial therapeutic or bioreagent applications. It is conceived that the plant expression systems have the advantages of low production costs, rapid scalability, and no risk of contamination with adventitious animal pathogens. A comparison of past plant and mammalian IgA expression attempts is shown in Table 1. The yield of produced sIgA has been reported these systems to range from 6 to 57.7 μg/g.

TABLE 1
Eukaryotic expression yields for extraction
and isolation of secretory IgA (sIgA)

| Expression | | Biomass Produced | Biomass |
|---|---|---|---|
| System | Antibody | (μg/g) | Type** |
| Tobacco | sIgA | 6-15.2 | Wet |
| Arabidopsis | IgG/sIgA hybrid | 57.7 | Leaf Tissue |
| Tobacco | sIgA | 32.5 | Leaf Tissue |
| HEK293 | sIgA | 29* | Cell Culture |
| CHO/dhfr-cells | sIgA | 25* | Cell Culture |

*Biomass calculated using 1 L = 1000 g water weight.
**Type of starting material used for extraction of expressed sIgA.

Other attempts propose higher yields; however, from the data available, these approaches only manage to produce monomeric IgA, not the fully assembled sIgA based on subsequent analysis. Other approaches have included expression of individual sIgA components throughout tissues of individual plants, followed by crossbreeding of the plants to create progeny containing all four component proteins. However, it is still unclear from this work how much antibody produced is actually the full assembled and bioactive sIgA. In addition, the expression yields demonstrated, even in the progeny are still below commercially feasible levels. Moreover, extraction from harvested leaves must be carried out within a very quick timeline, usually within twelve hours, to obtain fully assembled sIgA that is stable and maintains activity. Leaf tissue is notorious for rapid degradation of target protein. This presents obvious difficulties at pilot and production scales; the process is most practical under laboratory-scale experimental conditions in which leaves can be stored frozen to reduce the loss of bioactive protein prior to extraction and purification. Finally, attempts have followed Agrobacterium-mediated transformation with gene constructs containing all four genes each encoding LC, HC, JC, and SC, respectively, but still demonstrate a lack of expression yield and inability to accumulate fully assembled sIgA in a stable form.

Accordingly, there still remains a high need for feasible approaches to express immunoglobulins in Eukaryotic systems, and particularly sIgA, at commercially reasonable yields.

### SUMMARY OF THE INVENTION

The present invention relates to a method for production of fully assembled and complete secretory antibodies (sIgA) in eukaryotic cells, at levels two or more times higher than other industrial animal or plant (Eukaryote) expression systems. Secretory IgA compositions can be extracted and purified from these expression systems for use as therapeutics, prophylactic medicines, and diagnostics.

In general, expression vectors can include the following operably linked components that constitute a chimeric nucleic acid construct: a promoter from a host-specific protein storage gene, a signal peptide sequence capable of targeting to a protein storage organelle, such as a protein storage body, and a sequence encoding for one of the immunoglobulin components linked in translation frame with the signal peptide sequence. Host cells co-transformed with a plurality of nucleic acid constructs, each containing one of the immunoglobulin components with the same promoter and signal peptide, such that all requisite immunoglobulin components are introduced into the cell, subsequently express fully assembled Ig molecules. The constructs also each include a transcriptional termination region generally at the opposite end of the vector from the transcription initiation regulatory region.

In one or more embodiments, suitable plant-transformation vectors are designed for operation in plants, with associated upstream and downstream sequences for expression and protein assembly in plant protein storage bodies.

This expression system (referred to as ExpressTec) unexpectedly achieves commercially viable expression levels of fully assembled and bioactive secretory IgA antibodies. Advantageously, the system can store fully assembled sIgA products until extraction and purification at commercial production scale.

Thus, advantages of the invention include, without limitation, commercially feasible expression yield (>100 mg/kg) of secretory IgA expressed in a eukaryotic expression hosts capable of generating fully assembled and functional SIgA; pharmaceutical compositions containing sIgA for oral delivery for diseases including inflammatory conditions, infectious disease or auto-immune diseases; pharmaceutical compositions containing sIgA for topical delivery to treat diseases of the skin, lungs and nasal passages; and pharmaceutical compositions containing sIgA for parenteral delivery to treat diseases of the skin, lungs and nasal passages.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A show schematic representations of IgA and sIgA architectures for IgA1 isotype of IgA. dIgA=dimeric IgA; Short, thin connecting lines represent interchain disulfide bonds. Dark and light grey “lollipops” represent O- and N-linked oligosaccharides respectively.

FIG. 1B show schematic representations of IgA and sIgA architectures for IgA2 ml isotype of IgA. dIgA=dimeric IgA; Short, thin connecting lines represent interchain disulfide bonds. Dark and light grey “lollipops” represent O- and N-linked oligosaccharides respectively.

FIG. 1C show schematic representations of IgA and sIgA architectures for IgA2m2 isotype of IgA. dIgA=dimeric IgA; Short, thin connecting lines represent interchain disulfide bonds. Dark and light grey “lollipops” represent O- and N-linked oligosaccharides respectively.

FIG. 2 shows the protein sequences of the constant regions of the alpha heavy chains (HC) of IgA1, IgA2m1 and IgA2m2, and the protein sequence of the variable region that is common to all three alpha heavy chains.

FIG. 3 shows the protein sequence of the kappa light chain (LC) constant and variable region.

FIG. 4 shows the protein sequence of the joining chain (JC).

FIG. 5 shows the protein sequence of the secretory component (SC).

FIG. 6 shows schematic representations of the transformation constructs for each of the individual sIgA chains, as well as the construct for a selectable marker used for transformant selection. Gt 1, rice glutelin gene promoter (SEQ ID NO:16); NOS Terminator, nopaline synthase terminator from Agrobacterium tumefaciens (SEQ ID NO:17); Gns9, rice glucanase 9 gene (Gns9) promoter; Hygromycin-R, hygromycin B resistance gene; RAmy, rice amylase gene terminator;

FIG. 7 shows a flowchart overview of the transformation protocol used for the plant ExpressTec system.

FIG. 8 shows Western Blots that confirm the presence of all expressed chains and verify high-level expression of assembled sIgA and IgA in seed extracts of a rice transformant.

FIG. 9 shows analytical protein-L chromatography data that quantify the high expression level of IgA species in the ExpressTec system.

FIG. 10A shows an image from an SDS-Page gel from chromatographic separation of protein-L purified sIgA from IgA species using size exclusion chromatography (gel filtration chromatography);

FIG. 10B shows the chromatography data where representative Peak fractions from Size-Exclusion Chromatography show separation and high purity of secretory IgA and IgA species.

FIG. 10C shows an image of a gel where representative Peak fractions from Size-Exclusion Chromatography show separation and high purity of secretory IgA and IgA species.

FIG. 11A shows Western Blot images of the heavy (HC) and light (LC) chains from Size-Exclusion Chromatography that confirm the identity of sIgA and IgA in the protein-L purified, size-fractionated IgA samples.

FIG. 11B shows a Western Blot image from Size-Exclusion Chromatography that confirm the identity of sIgA and IgA in the protein-L purified, size-fractionated IgA samples.

FIG. 12 shows a graph of in vitro neutralization of the sIgA101 target, soluble Tumor Necrosis Factor alpha, by protein-L purified, size-fractionated sIgA101 species in an in vitro L929 cell survival assay (TNF-alpha neutralization assay).

FIG. 13 shows a graph demonstrating in vitro neutralization of the sIgA101 target, soluble Tumor Necrosis Factor alpha, by protein-L purified, size-fractionated sIgA101 species in an in vitro Human colonic epithelial cell monolayer permeability assay, by measurement of transepithelial electrical resistance (TEER).

FIG. 14 shows the results from the in vivo functional validation of purified sIgA101 based upon clinical scores in a mouse DSS model.

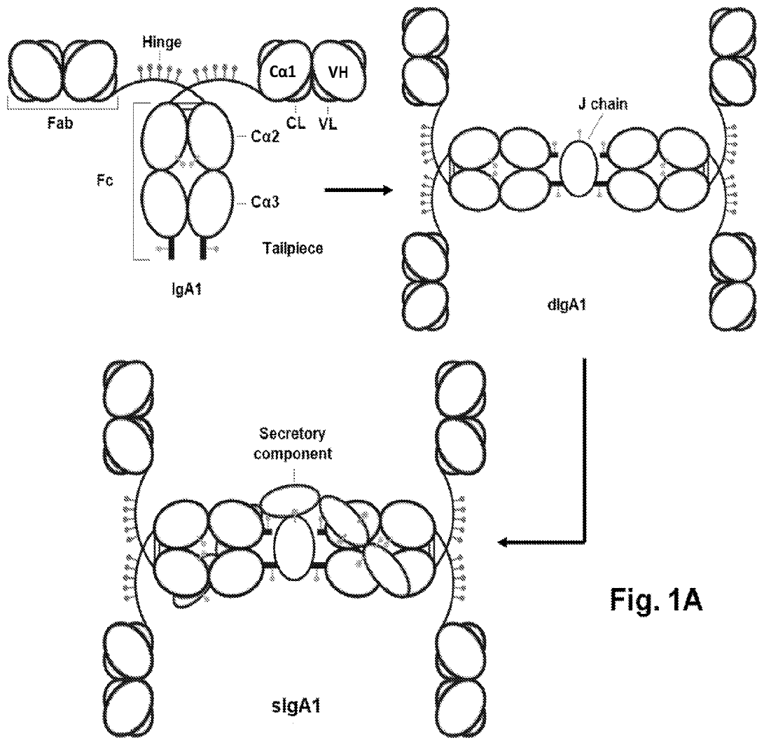

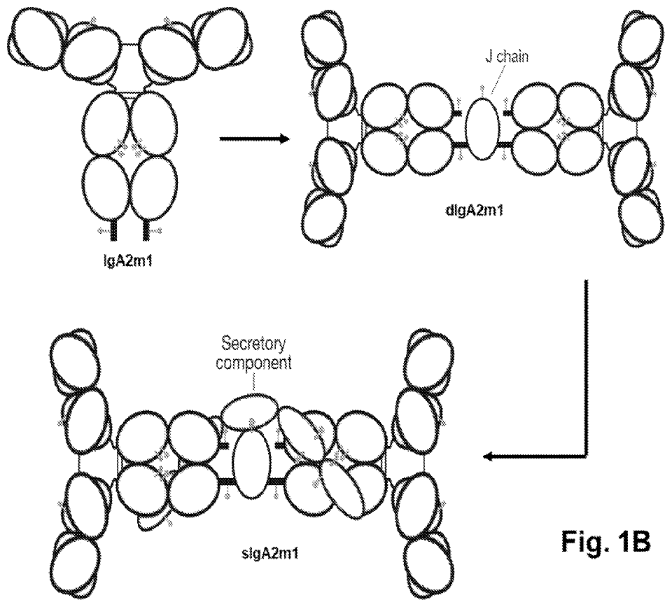

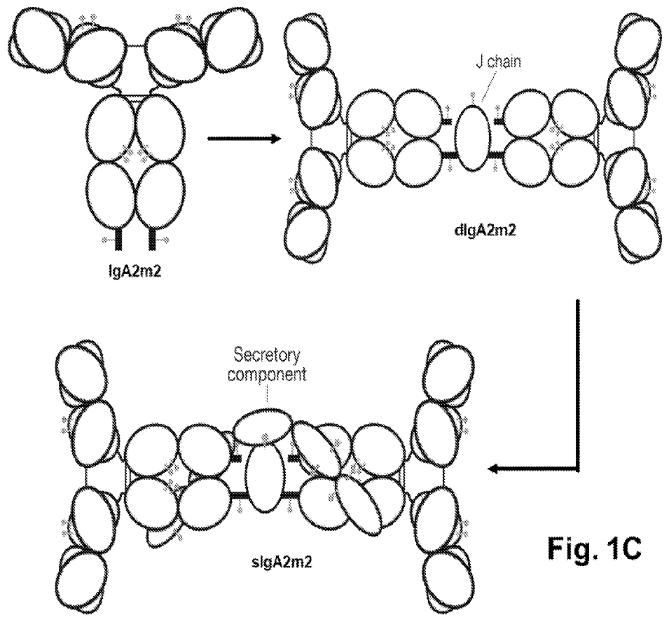

### DETAILED DESCRIPTION

As noted, recombinant sIgA production in plants or other eukaryotic systems requires the co-expression of four transcriptional units respectively encoding the light chain (LC), heavy chain (HC), joining chain (JC), and secretory component (SC), and subsequent intracellular assembly of the expressed components into the functional protein.

In one aspect, methods of producing recombinant sIgA protein in a eukaryotic expression systems are described. The methods generally comprise co-transforming a eukaryotic cell with at least four different nucleic acid constructs, each comprising a respective transcriptional unit encoding the light chain (LC), heavy chain (HC), joining chain (JC), or secretory component (SC) of sIgA. Advantageously, each nucleic acid construct comprises the same promoter and signal sequence, such that each of the LC, HC, JC, and SC polypeptides will be targeted to the same organelle of the cell for expression and assembly. In one or more embodiments, each nucleic acid construct comprises a promoter from a protein storage gene that is operably linked to a DNA sequence that encodes for a protein storage-specific signal sequence capable of targeting a polypeptide linked thereto to a protein storage organelle of the eukaryotic cell, and a second DNA sequence, linked in translation frame with the signal sequence, wherein the second DNA sequence is a transcriptional unit encoding the light chain (LC), the heavy chain (HC), the joining chain (JC), or the secretory component (SC) of sIgA. In one or more embodiments, the transcriptional unit encoding for the sIgA components comprises codon optimized DNA sequences.

In one or more embodiments, the second DNA sequence encodes for the heavy chain of sIgA, for example, encodes for a heavy chain constant region selected from the group consisting of SEQ ID NO:1, SEQ ID NO:2, SEQ ID NO: 3, and conservatively modified variants or homologs thereof; and a heavy chain variable region selected from the group consisting of SEQ ID NO:4, SEQ ID NO:9, and conservatively modified variants or homologs thereof. In one or more embodiments, the DNA sequence encoding for the heavy chain of sIgA (both variable and constant regions) comprises, consists essentially, or even consists of SEQ ID NO:12, or conservatively modified variants or homologs thereof. In one or more embodiments, the second DNA sequence encodes for a light chain constant region selected from the group consisting of SEQ ID NO: 5, and conservatively modified variants or homologs thereof; and a light chain variable region selected from the group consisting of SEQ ID NO:6, SEQ ID NO:10, SEQ ID NO:11, and conservatively modified variants or homologs thereof. In one or more embodiments, the DNA sequence encoding for the light chain of sIgA (both variable and constant regions) comprises, consists essentially, or even consists of SEQ ID NO:13, or conservatively modified variants or homologs thereof. In one or more embodiments, the second DNA sequence encodes for a joining chain selected from the group consisting of SEQ ID NO:7 and conservatively modified variants or homologs thereof. In one or more embodiments, the DNA sequence encoding for the J chain of sIgA comprises, consists essentially, or even consists of SEQ ID NO:14, or conservatively modified variants or homologs thereof. In one or more embodiments, the second DNA sequence encodes for a secretory component selected from the group consisting of SEQ ID NO:8 conservatively modified variants or homologs thereof. In one or more embodiments, the DNA sequence encoding for the secretory component of sIgA comprises, consists essentially, or even consists of SEQ ID NO:15, or conservatively modified variants or homologs thereof.

As noted, the expression constructs comprise a specific promoter/signal sequence capable of targeting a polypeptide linked thereto (e.g., the expressed sIgA components) to a protein storage organelle of the eukaryotic host expression cell, and thus, the above sequences are each linked in translation frame with a respective promoter/signal sequence. In one or more embodiments, the protein storage gene and/or signal peptide are native to the eukaryotic cell. In other embodiments, the protein storage gene and signal peptide are heterologous to the eukaryotic cell. In one or more embodiments, the signal sequence encodes a rice glutelin signal sequence, for example, encodes for a rice glutelin comprising, consisting essentially, or even consisting of SEQ ID NO:18 or conservatively modified variants or homologs thereof. In one or more embodiments, both the promoter and signal sequence encodes a glutelin (Gt1) promoter and signal sequence and has a nucleic acid sequence selected from the group consisting of SEQ ID NO:16 and conservatively modified variants or homologs thereof. Homologous seed protein storage sequences can also be used. Likewise, codon-optimized variants of the exemplified sequences can be applied directly in other plant species as discussed in more detail below (e.g., a codon-optimized version of rice Gt1 promoter can be applied in wheat, barley, etc.). Various suitable terminator sequences can be used with an exemplary sequence being selected from the group consisting of SEQ ID NO:17 and conservatively modified variants or homologs thereof.

Preferred nucleic acid constructs are exemplified in the working examples. In one or more embodiments, an exemplified expression construct for expressing the sIgA heavy chain in a eukaryotic expression system comprises, consists essentially, or even consists of SEQ ID NO:19 or conservatively modified variants or homologs thereof. In one or more embodiments, an exemplified expression construct for expressing the sIgA light chain in a eukaryotic expression system comprises, consists essentially, or even consists of SEQ ID NO:20 or conservatively modified variants or homologs thereof. In one or more embodiments, an exemplified expression construct for expressing the sIgA J chain in a eukaryotic expression system comprises, consists essentially, or even consists of SEQ ID NO:21 or conservatively modified variants or homologs thereof. In one or more embodiments, an exemplified expression construct for expressing the sIgA secretory component in a eukaryotic expression system comprises, consists essentially, or even consists of SEQ ID NO:22 or conservatively modified variants or homologs thereof.

In one or more embodiments, the eukaryotic cell is additionally transformed with a nucleic acid construct comprising a selectable marker, which is preferably driven by the same promoter and signal peptide used for the sIgA components. Alternatively, the selectable marker may be driven by a different promoter and signal peptide.

Transformation is preferably carried out using microprojectile bombardment. In one or more embodiments, each microparticle or nanoparticle used for bombardment comprises each of the four nucleic acid constructs. In one or more embodiments, the nucleic acid construct comprising the selectable marker is further included on the particle. In other words, all of the nucleic acid constructs are preferably included on each of the particles used for bombardment. In one or more embodiments, extensive rounds of bombardment are carried out on each calli, e.g., at least a dozen rounds of bombardment.

In one aspect, methods of expressing sIgA protein in plant seeds are described. The methods generally comprise co-transforming a plant cell with at least four different nucleic acid constructs, each comprising a respective transcriptional unit encoding the light chain (LC), heavy chain (HC), joining chain (JC), or secretory component (SC) of sIgA. The nucleic acid constructs each comprise a sequence encoding for a seed storage promoter protein, operably linked to a signal sequence capable of targeting a polypeptide linked thereto to a plant seed endosperm cell, and a second DNA sequence, linked in translation frame with the signal sequence, wherein the second DNA sequence is a transcriptional unit encoding the light chain (LC), the heavy chain (HC), the joining chain (JC), or the secretory component (SC) of sIgA. In one or more embodiments, the plant cell is additionally transformed with a nucleic acid construct comprising a selectable marker, which is preferably driven by the same promoter and signal peptide used for the sIgA components.

In one or more embodiments, the methods include growing a plant from the transformed plant cell for a time sufficient to produce seeds containing the sIgA components, preferably, including fully assembled and bioactive sIgA. The seeds can be harvested from the plant, wherein assembled sIgA constitutes at least 0.1% dry seed weight of the harvested seeds, preferably at least 0.3% dry seed weight. In one or more embodiments, sIgA makes up at least about 25% of the total soluble protein product in the seeds.

It will be appreciated that the particular techniques discussed herein in regard to sIgA may be applied to other multigenic proteins, including other immunoglobulins. For ease of reference, the discussions herein use sIgA as an exemplary embodiment. In one or more embodiments, the techniques of the invention permit achievement of expression yields of at least about 100 mg/kg preferably at least about 200 mg/kg, more preferably at least about 500 mg/kg, more preferably at least about 1 g/kg, more preferably at least about 2 g/kg, more preferably at least about 3 g/kg, even more preferably from about 3 g/kg to about 18 g/kg, even more preferably from about 3.5 g/kg to about 16 g/kg (as measured from the amount of protein extractable from 1 kg of biomass, i.e., flour).

The above-described approaches facilitate site-specific expression of the desired proteins, in contrast to previous approaches that have indiscriminately expressed the recombinant protein throughout various tissues of transgenic plants. In one or more embodiments, the approach preferably utilizes promoters and signal peptides specific to the host cell, instead of selecting sequences specific to the protein that is targeted for expression, or using other generic viral promoter systems.

In some embodiments, the transgenic plant may further comprise a nucleic acid that encodes at least one transcription factor such as opaque 2 (O2), prolamin box factor (PBF), and the rice endosperm bZIP protein (Reb). In one or more embodiments, described herein are transgenic plants which comprise the heterologous nucleic acid coding sequence for one or more plant transcription factors operably linked to a seed specific promoter, wherein expression of the transcription factor(s) in a plant cell is effective to activate transcription of a DNA sequence encoding for light chain (LC), the heavy chain (HC), the joining chain (JC), or the secretory component (SC) of sIgA operably linked to the seed specific promoter with which the one or more transcription factors interact.

As noted, the expression vectors comprise protein storage organelle-specific promoters. In the case of transgenic plants, the expression vectors comprise seed-specific promoters. The transcription regulatory or promoter region of the heterologous nucleic acid construct is preferably a seed-specific promoter, for example, a promoter capable of directing expression of a gene product under its control, which is specific to the seed embryo, aleurone, outer layer of the endosperm or center of the endosperm; or a promoter capable of directing expression of a gene product under its control, which is specific to starch or protein synthesis. In general, the expression construct preferably comprises a promoter that exhibits specifically upregulated activity (greater than 25%) during seed maturation. Promoters for plant systems are typically derived from cereals such as rice, barley, wheat, maize, oat, rye, corn, millet, triticale or sorghum. Examples of such promoters include the maturation-specific promoter region associated with one of the following maturation-specific monocot plant storage proteins: rice glutelins, oryzins, and prolamines, barley hordeins, wheat gliadins and glutelins, maize zeins and glutelins, oat glutelins, and sorghum kafirins, millet pennisetins, and rye secalins. Some promoters suitable for expression in maturing seeds include glutelin (Gt-1) promoter which effects gene expression in the outer layer of the endosperm and a globulin (Glb) promoter which effects gene expression in the center of the endosperm. Promoter sequences for regulating transcription of operably linked coding sequences include naturally-occurring promoters, or regions thereof capable of directing seed-specific transcription, and hybrid promoters, which combine elements of more than one promoter.

In some cases, the promoter is derived from the same plant species as the plant in which the nucleic acid construct is to be introduced.

Alternatively, a seed-specific promoter from one type of plant may be used regulate transcription of a gene coding sequence from a different plant. Numerous types of appropriate expression vectors, and suitable regulatory sequences are known in the art for a variety of plant host cells. In general, the transcriptional and translational regulatory sequences may include, but are not limited to, promoter sequences, ribosomal binding sites, transcriptional start and stop sequences, translational start and stop sequences, and enhancer or activator sequences. For example, although data are presented for the ectopic expression of fully functional/assembled sIgA in rice (Oryza sps.), it is contemplated that the rice G1b promoter linked to the rice Gt1 leader may be used in other Gramineae genera such as barley and wheat, and further that similar levels of protein expression is would be achieved given that the codons are optimized for the given host species. Those skilled in the art would appreciate that the information exemplified in rice can be applied to other Gramineae genera to achieve similar results without undue experimentation. A particular, non-limiting example of applying this information in barley is provided in the Examples.

Effective seed-inducible or seed-regulated transcriptional initiation regions (e.g., promoters) may be isolated from various seed tissues and/or at various stages of seed development. Promoters from seed tissue specific genes are suitable for use herein. More specifically, representative seed-associated promoters for use in the invention include the promoters from the rice glutelin multigene family, Gt1, Gt2, Gt3, GluA-3, and GluB-1. Promoter regions for these genes are described, for example under GenBank Accession Nos. D26365 and D26364 (rice glutelin gene), GenBank Accession No. X54313 (rice GluA-3 gene), GenBank Accession No. Y00687 (rice glutelin gene), GenBank Accession No. X54193 (rice Glu-B gene); GenBank Accession No. X54192 (rice GluB-2 gene); GenBank Accession No. X54314 (rice GluB-1 gene); GenBank Accession No. L36819 M28157 (rice Gt2 gene); GenBank Accession No. M28158 (rice Gt3 gene); GenBank Accession No. M28156 (rice Gt1 gene); GenBank Accession Nos. D26363, D26366+D26367, D26368 and D26369 (rice glutelin gene); GenBank Accession No. D00584 (rice prepro-glutelin gene); GenBank Accession No. X52153 (rice glutelin gene). In general, these promoters are active during seed development and direct endosperm-specific expression.

Other suitable seed-associated promoters include the promoter regions from the rice prolamin gene (GenBank Accession No. D73384); the barley B22EL8 gene promoter, which directs expression in immature aleurone layers; the promoter for the barley LTp gene (GenBank Accession No. X57270); the barley O-amylase (GenBank Accession No. X52321 and M36599) and O-glucanase gene promoters, such as the barley G1b gene promoter (GenBank Accession No. X56775); the barley CMd gene promoter (GenBank Accession No. X13198), and promoters from the barley hordein gene family of seed storage proteins, such as B-, C-, and D-hordein genes (hordein B1 gene promoter, GenBank Accession No. X87232; barley hordein C promoter, GenBank Accession No. M36941; barley hor1-17 gene, GenBank Accession No. X60037). Hordein gene promoters such as the Hor3 gene promoter (GenBank Accession No. X84368) direct the specific expression of the corresponding genes in the endosperm. Additional seed-induced promoters for use in the invention are the maize zein gene promoter and promoters from wheat glutenin genes. Representative wheat glutenin gene sequences as sources for promoters for use in practicing the present invention include GenBank Accession Nos. U86028, U86029, and U86030. The sequences of the above-described promoters, and/or the structural sequences from which such promoters may obtained, are expressly incorporated by reference herein.

Seed-associated corn promoters also find use in the present invention. For example, the corn 02-opaque 2 gene promoter (GenBank Accession No. M29411); the corn Sh2-shrunken 2 gene promoter (GenBank Accession No. S48563); the Bt2-brittle 2 gene promoter; and the Zp1 zein gene promoter, all of which induce endosperm-specific expression. Additional examples include the Agp1 and Agp2 gene promoters, which are embryo-specific promoters. The sequences of the above-described promoters, and/or the structural sequences from which such promoters may obtained, are expressly incorporated by reference herein.

Any of the above promoters may also be obtained from an alternative species. For example, a promoter such as the Gt1 gene promoter from rice may be isolated from other cereal-derived nucleic acid containing extracts, e.g., wheat, oat, or the like, using conventional hybridization techniques known in the art. Regardless, promoters for use in embodiments of the invention are preferentially expressed in plant seed tissue, such that methods described herein are directed toward the localization of proteins in an endosperm cell, in some embodiments an endosperm-cell organelle, such as a protein storage body.

In addition to encoding the protein of interest, the expression cassette or heterologous nucleic acid construct includes DNA encoding a signal peptide that allows processing and translocation of the protein, as appropriate. Exemplary signal sequences are those sequences associated with the monocot maturation-specific genes: glutelins, prolamines, hordeins, gliadins, glutenins, zeins, albumin, globulin, AOP glucose pyrophosphorylase, starch synthase, branching enzyme, Em, and lea. Exemplary sequences encoding a signal peptide for a protein storage body are identified herein, and include: bx7 signal peptide sequence (SEQ ID NO:23), Glub-2 signal peptide sequence (SEQ ID NO:24), Gt3 signal peptide sequence (SEQ ID NO:25), Glub-1 signal peptide sequence (SEQ ID NO:26), prolamin signal peptide sequence (SEQ ID NO:27), Rice cysteine peptidase signal peptide sequence (SEQ ID NO:28), D-Hordein signal peptide sequence (SEQ ID NO:29).

Another exemplary class of signal/targeting/transport sequences are sequences effective to promote secretion of heterologous protein from aleurone cells during seed germination, including the signal sequences associated with alpha-amylase, protease, carboxypeptidase, endoprotease, ribonuclease, DNase/RNase, (1-3)-beta-glucanase, (1-3)(1-4)-beta-glucanase, esterase, acid phosphatase, pentosamine, endoxylanase, β-xylopyranosidase, arabinofuranosidase, beta-glucosidase, (1-6)-beta-glucanase, perioxidase, and lysophospholipase.

Since many protein storage proteins are under the control of a maturation-specific promoter, and this promoter is operably linked to a signal sequence for targeting to a protein body, the promoter and signal sequence can be isolated from a single protein-storage gene, then operably linked to an immunoglobulin protein in the chimeric gene construction. One exemplary promoter-signal sequence combination is exemplified here, in which the promoter and signal sequence both come from the rice Gt1 gene regulatory region. Alternatively, the promoter and leader sequence may be derived from different genes. One exemplary promoter-signal sequence combination is the rice G1b promoter linked to the rice Gt1 leader sequence (SEQ ID NO:16 and 18).

Expression vectors or heterologous nucleic acid constructs designed for operation in plants further comprise companion sequences upstream and downstream to the expression cassette. The transcription termination region may be taken from a gene where it is normally associated with the transcriptional initiation region or may be taken from a different gene. Exemplary transcriptional termination regions include the NOS terminator from Agrobacterium Ti plasmid and the rice α-amylase terminator.

Polyadenylation tails may also be added to the expression cassette to optimize high levels of transcription and proper transcription termination, respectively. Polyadenylation sequences include, but are not limited to, the Agrobacterium octopine synthetase signal, or the nopaline synthase of the same species.

As noted, the cells can also be co-transformed with a nucleic acid encoding for a selectable marker. Suitable selectable markers for selection in plant cells include, but are not limited to, antibiotic resistance genes, such as, kanamycin (nptII), G418, bleomycin, hygromycin, chloramphenicol, ampicillin, tetracycline, and the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance; and a methotrexate resistant DHFR gene.

The particular marker gene employed is one which allows for selection of transformed cells as compared to cells lacking the nucleic acid which has been introduced. The selectable marker gene is one which facilitates selection at the tissue culture stage, e.g., a kanamyacin, hygromycin or ampicillin resistance gene.

The vector sequences are preferably stably transformed, and may be integrated into the host genome for constitutive expression.

In some embodiments, the host cell is a monocot plant cell, such as, for example, a monocot endosperm cell. Exemplary plants of monocot origin include members of the taxonomic family known as the Gramineae. This family includes all members of the grass family of which the edible varieties are known as cereals or grains. The cereals include a wide variety of species such as wheat (Triticum sps.), rice (Oryza sps.), barley (Hordeum sps.), oats (Avena sps.), rye (Secale sps.), corn (Zea sps.), and millet (Pennisettum sps.). In one embodiment of the invention, preferred family members are rice, wheat and barley.

Plant cells or tissues derived from the members of the family may be transformed with expression vectors described herein. The transgenic plant cells are cultured in medium containing the appropriate selection agent to identify and select for plant cells which express the heterologous nucleic acid sequence. After plant cells that express the heterologous nucleic acid sequence are selected, whole plants are regenerated from the selected transgenic plant cells. Plant cells that grow on or in the selective media are typically transferred to a fresh supply of the same media and cultured again. The explants are then cultured under regeneration conditions to produce regenerated plant shoots. After shoots form, the shoots are transferred to a selective rooting medium to provide a complete plantlet. The plantlet may then be grown to provide seed, cuttings, or the like for propagating the transformed plants. The method provides for efficient transformation of plant cells and regeneration of transgenic plants, which can produce sIgA.

Genetic crosses be subsequently carried out using conventional plant breeding techniques. However, it will be appreciated that the methods of the invention permit expression and assembly of functional sIgA in a given plant cell (and subsequent plant), without the need for crossing. In other words, the first generation plants are co-transformed with all four components necessary to express the full, bioactive sIgA molecule and at commercially-relevant expression yield. Crossing can be carried out if desired to further enhance expression yields.

In one example of this approach, a first stable transgenic plant line is generated where the plants express sIgA under the control of a seed-specific promoter. A number of such lines may be generated with varying levels of sIgA expression. The plants are crossed with a second transgenic plant line that expresses a sIgA under the control of a seed-specific promoter. The resulting cross (F2) has a higher expression level of sIgA in one or more particular seed tissues, dependent upon the promoter used.

The expression of sIgA protein may be confirmed using standard analytical techniques such as Western blot, ELISA, PCR, HPLC, NMR, or mass spectroscopy, together with assays for a biological activity specific to the particular protein being expressed.

A plant seed product prepared from the harvested seeds is also provided in the present disclosure. Preferably, the total amount of IgA (monomeric and sIgA) is at least about 10%, more preferably at least about 25%, and even more preferably at least about 35% of the total soluble protein in the seed product. Preferably, the sIgA protein constitutes at least about 5% of the total soluble protein in the seed product, more preferably at least about 10%, and most preferably at least about 25%. As shown in the data, the expression of sIgA proteins in rice grains represent at least about 25% of total soluble protein. In one or more embodiments, the sIgA is extractable from rice flour.

Embodiments of the invention are also concerned with a purified sIgA protein recombinantly produced at an expression yield of greater than 100 mg/kg, and up to about 16 g/kg of secretory IgA in a eukaryotic expression system, such as a plant seed expression system. The present disclosure also provides compositions comprising immunoglobulin proteins produced recombinantly in the described expression systems, and methods of making such compositions.

In one or more embodiments, immunoglobulins produced according to the invention, may be administered to a subject in substantially unpurified form (i.e., at least 10-20% of the composition comprises plant material), or the immunoglobulin protein may be isolated or purified from a product of the mature seed (e.g., a flour, extract, malt or whole seed composition, etc.) and formulated for delivery to a subject. The immunoglobulin can be purified from the seed product by a mode including grinding, filtration, heat, pressure, salt extraction, evaporation, or chromatography.

In some embodiments, a seed composition containing a flour, extract, or malt obtained from mature monocot seeds and one or more seed-produced immunoglobulin(s) protein in unpurified form is provided. Isolating the immunoglobulin(s) protein from the flour can entail forming an extract composition by milling seeds to form a flour, extracting the flour with an aqueous buffered solution, and optionally, further treating the extract to partially concentrate the extract and/or remove unwanted components. In a preferred method, mature monocot seeds, such as rice seeds, are milled to a flour, and the flour then suspended in saline or in a buffer, such as Phosphate Buffered Saline (“PBS”), ammonium bicarbonate buffer, ammonium acetate buffer or Tris buffer. A volatile buffer or salt, such as ammonium bicarbonate or ammonium acetate may obviate the need for a salt-removing step, and thus simplify the extract processing method.

In some embodiments, the level of protein expressed in a transgenic plant is assessed from a crude extract or substantially unpurified composition from the plant seed. In some embodiments, a grain or milled grain or flour composition, an extract composition, or malt composition obtained from mature monocot seeds is produced in substantially unpurified form. The immunoglobulin(s) protein may be present in an amount between about 0.5 and 3 grams protein/kg total soluble protein. For a grain composition, the level of immunoglobulin(s) protein present may be between 0.05 to 0.3% of total seed weight. For an extract composition, the immunoglobulin(s) protein may be concentrated to more of the total extract weight.

The flour suspension is incubated with shaking for a period typically between 30 minutes and 4 hours, at a temperature between 4-55° C., The resulting homogenate is clarified either by filtration or centrifugation. The clarified filtrate or supernatant may be further processed, for example by ultrafiltration or dialysis or both to remove contaminants such as lipids, sugars and salt. Finally, the material may be dried, e.g., by lyophilization, to form a dry cake or powder. The extract combines advantages of high protein yields, essentially limiting losses associated with protein purification.

In general, the protein once produced in a product of a mature seed can be further purified by standard methods known in the art, such as by filtration, affinity column, gel electrophoresis, and other such standard procedures. The purified protein can then be formulated as desired for delivery to a subject, such as a human or an animal. The invention finds use for both human and veterinary applications. More than one protein can be combined for the therapeutic human or veterinary formulation. The protein may be purified and used in biomedical applications requiring a non-food administration of the protein.

In one or more embodiments, the isolated or purified protein may be used in a therapeutic or prophylactic composition. Such compositions often include carriers or excipients. The term carrier is used herein to refer to diluents, excipients, vehicles, and the like, in which the immunoglobulin(s) may be dispersed for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would naturally be selected to minimize any degradation of the immunoglobulin(s) or other agents and to minimize any adverse side effects in the subject, as would be well known to one of skill in the art. Pharmaceutically-acceptable ingredients include those acceptable for veterinary use as well as human pharmaceutical use, and will depend on the route of administration. For example, compositions suitable for administration via injection are typically solutions in sterile isotonic aqueous buffer. Exemplary carriers include aqueous solutions such as normal (n.) saline (˜0.9% NaCl), phosphate buffered saline (PBS), sterile water/distilled autoclaved water (DAW), aqueous dextrose solutions, aqueous glycerol solutions, ethanol, normal allantoic fluid, various oil-in-water or water-in-oil emulsions, as well as dimethyl sulfoxide (DMSO) or other acceptable vehicles, and the like.

The composition can comprise a therapeutically effective amount of immunoglobulin(s) dispersed in the carrier. As used herein, a “therapeutically effective” amount refers to the amount that will elicit the biological or medical response of a tissue, system, or subject that is being sought by a researcher or clinician, and in particular elicit some desired protective or therapeutic effect as against the infection. One of skill in the art recognizes that an amount may be considered therapeutically “effective” even if the condition is not totally eradicated or prevented, but it or its symptoms and/or effects are improved or alleviated partially in the subject. In some embodiments, the composition will comprise from about 5% to about 95% by weight of immunoglobulin(s) described herein, and preferably from about 30% to about 90% by weight of immunoglobulin(s), based upon the total weight of the composition taken as 100% by weight. In some embodiments, combinations of more than one type of the described immunoglobulin(s) can be included in the composition.

Such compositions can comprise a formulation for the type of delivery intended. Delivery types can include, e.g. parenteral, enteric, inhalation, intranasal or topical delivery. Parenteral delivery can include, e.g. intravenous, intramuscular, or suppository. Enteric delivery can include, e.g. oral administration of a pill, capsule, or other formulation made with a non-nutritional pharmaceutically-acceptable excipient, or a composition with a nutrient from the transgenic plant, for example, in the grain extract in which the protein is made, or from a source other than the transgenic plant. Such nutrients include, for example, salts, saccharides, vitamins, minerals, amino acids, peptides, and proteins other than the immunoglobulin protein. Intranasal and inhalant delivery systems can include spray or aerosol in the nostrils or mouth. Topical delivery can include, e.g. creams, topical sprays, or salves. In some embodiments, the composition is substantially free of contaminants of the transgenic plant, preferably containing less than 20% plant material, more preferably less than 10%, and most preferably, less than 5%. In some embodiments, the preferable route of administration is enteric, and preferably the composition is non-nutritional. Various stabilized formulations for immunoglobulin-type proteins are described in detail in co-pending PCT/US2019/049709, filed Sep. 5, 2019, incorporated by reference in its entirety herein.

As will be understood by those of skill in the art, in some cases it may be advantageous to use an immunoglobulin protein-encoding nucleotide sequences possessing non-naturally occurring codons. Codons preferred by a particular eukaryotic host can be selected, for example, to increase the rate of immunoglobulin protein expression or to produce recombinant RNA transcripts having desirable properties, such as a longer half-life, than transcripts produced from naturally occurring sequence. As an example, it has been shown that codons for genes expressed in rice are rich in guanine (G) or cytosine (C) in the third codon position. Changing low G+C content to a high G+C content has been found to increase the expression levels of foreign protein genes in barley grains. The protein encoding genes can be synthesized based on the rice gene codon bias along with the appropriate restriction sites for gene cloning. These “codon-optimized” genes are then linked to regulatory/secretion sequences for seed-directed expression and these chimeric genes then inserted into the appropriate plant transformation vectors.

Heterologous nucleic acid constructs may include the coding sequence for an immunoglobulin protein (i) in isolation; (ii) in combination with additional coding sequences; such as fusion protein or signal peptide, in which the immunoglobulin protein coding sequence is the dominant coding sequence; (iii) in combination with non-coding sequences, such as introns and control elements, such as promoter and terminator elements or 5′ and/or 3′ untranslated regions, effective for expression of the coding sequence in a suitable host; and/or (iv) in a vector or host environment in which the immunoglobulin protein coding sequence is a heterologous gene.

Depending upon the intended use, an expression construct may contain the nucleic acid sequence encoding the entire immunoglobulin component protein, or a portion thereof. For example, where immunoglobulin protein sequences are used in constructs for use as a probe, it may be advantageous to prepare constructs containing only a particular portion of the immunoglobulin protein encoding sequence, for example a sequence which is discovered to encode a highly conserved immunoglobulin protein region.

### Definitions

“Heterologous DNA” refers to DNA which has been introduced into the host eukaryotic cells from another source, or which can be from a plant source, including the same plant source, but which is under the control of a promoter that does not normally regulate expression of the heterologous DNA.

“Heterologous protein” is a protein encoded by a heterologous DNA.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

A cell, tissue, organ, or plant into which a heterologous nucleic acid construct comprising the coding sequence for an immunoglobulin component has been introduced is considered transformed, transfected, or transgenic. A transgenic or transformed cell or plant also includes progeny of the cell or plant and progeny produced from a breeding program employing such a transgenic plant as a parent in a cross and exhibiting an altered phenotype resulting from the presence of the coding sequence for the immunoglobulin. Hence, a plant of the present disclosure will include any plant which has a cell containing introduced nucleic acid sequences, regardless of whether the sequence was introduced into the plant directly through transformation means or introduced by generational transfer from a progenitor cell which originally received the construct by direct transformation.

References herein to “conservatively modified variants or homologs” refers to variants of the disclosed sequences which have been modified from the exact sequence shown but which nonetheless express a functionally equivalent protein, or to sequences of a similar structure and evolutionary origin to the same gene or protein sequence in another species, and accordingly having equivalent functional characteristics. Amino acid or nucleotide sequences are said to be homologous when exhibiting a certain level of similarity. Whether two homologous sequences are closely related or more distantly related is indicated by percent (%) identity or sequence identity. which is high or low respectively. Thus, the sequence “identity” or amino acid “identity” are used herein to describe the sequence relationships between two or more nucleic acid or amino acid sequences when aligned for maximum correspondence over a specified comparison window. In order to determine the percent identity of two amino acid sequences or of two nucleic acid sequences, the sequences are aligned for optimal comparison purposes. In order to optimize the alignment between the two sequences gaps may be introduced in any of the two sequences that are compared. After alignment, the number of matched positions (i.e., positions where the identical nucleic acid base or amino acid residue occurs in both sequences) is determined and then divided by the total number of positions in the comparison window. This result is then multiplied by 100 to calculate the percentage of sequence or amino acid identity. It will be appreciated that a sequence having a certain % of sequence identity to a reference sequence does not necessarily have to have the same total number of nucleotides or amino acids. Thus, a sequence having a certain level of “identity” includes sequences that correspond to only a portion (i.e., 5′ non-coding regions, 3′ non-coding regions, coding regions, etc.) of the reference sequence. Preferably in the case of conserved sequences, sequence identity of conservatively modified variants or homologs for amino acids will be at least 85%, preferably at least 90%, and in some cases at least 95%. In the case of variable sequences, sequence identity of conservatively modified variants or homologs for amino acids will be at least 65%, preferably at least 70%, and in some cases at least 73%. For nucleic acid sequence, sequence identity will be at least 75%, for example at least 80%, for example at least 85%, for example at least 90%, for example at least 95% in the case of homologs or conservatively modified variants.

The term “transgenic plant” refers to a plant that has incorporated exogenous nucleic acid sequences, i.e., nucleic acid sequences which are not present in the native (“untransformed”) plant or plant cell. Thus, a plant having within its cells a heterologous polynucleotide is referred to herein as a “transgenic plant.” The heterologous polynucleotide is preferably stably integrated into the genome, but can be extra-chromosomal. The polynucleotide of the present disclosure is preferably stably integrated into the genome such that the polynucleotide is passed on to successive generations. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Transgenic” is used herein to include any cell, cell line, callus, tissue, plant part or plant, the genotype of which has been altered by the presence of heterologous nucleic acids including those transgenics initially so altered as well as those created by sexual crosses or asexual reproduction of the initial transgenics.

The terms “transformed” or “stably transformed” with reference to a plant cell means the plant cell has a non-native (heterologous) nucleic acid sequence integrated into its genome which is maintained through two or more generations. Stably transformed cells exhibit constitutive expression of the introduced sequence(s).

The term “expression” with respect to a protein or peptide refers to the process by which the protein or peptide is produced based on the nucleic acid sequence of a gene. The process includes both transcription and translation. The term “expression” may also be used with respect to the generation of RNA from a DNA sequence.

The term “introduced” in the context of inserting a nucleic acid sequence into a cell, means “transfection,” or “transformation” or “transduction” and includes the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

By “host cell” is meant a cell containing a vector and supporting the replication and/or transcription and/or expression of the heterologous nucleic acid sequence according to the invention.

A “plant cell” refers to any cell derived from a plant, including undifferentiated tissue (e.g., callus) as well as plant seeds, pollen, propagules, embryos, suspension cultures, meristematic regions, leaves, roots, shoots, gametophytes, sporophytes and microspores.

The term “mature plant” refers to a fully differentiated plant.

The term “seed product” includes, but is not limited to, seed fractions such as de-hulled whole seed, a flour composition (seed that has been de-hulled by milling and ground into a powder) a seed extract composition, in some embodiments, a protein extract (where the protein fraction of the flour has been separated from the carbohydrate fraction), a malt composition (including malt extract or malt syrup) and/or a purified protein fraction derived from the transgenic grain.

The term “biological activity” refers to any biological activity typically attributed (native) to that protein by those of skill in the art.

The term “non-nutritional” refers to a pharmaceutically acceptable excipient which does not as its primary effect provide nutrition to the recipient. The excipient may provide one of the following services to an enterically delivered formulation, including acting as a carrier for a therapeutic protein, protecting the protein from acids in the digestive tract, providing a time-release of the active ingredients being delivered, or otherwise providing a useful quality to the formulation in order to administer to the patient the immunoglobulin protein.

“Monocot seed components” refers to carbohydrate, protein, and lipid components extractable from monocot seeds, typically mature monocot seeds.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

A “signal sequence” is an N- or C-terminal polypeptide sequence which is effective to localize the peptide or protein to which it is attached to a selected intracellular or extracellular region. In some embodiments, the signal sequence targets the attached peptide or protein to a location such as an endosperm cell, in certain embodiments, an endosperm-cell organelle, such as an intracellular vacuole or other protein storage body, chloroplast, mitochondria, or endoplasmic reticulum, or extracellular space, following secretion from the host cell.

“Plant-derived” means that the source of the ingredient is a plant.

“Dry weight percent” or “% dry weight” or “percent seed dry weight” means, for example, a protein-yield of grams immunoglobulin per kilogram of dry seeds. For example, 1% seed dry weight of rice-expressed immunoglobulin means that 1 kilogram of rice grains yields 10 grams of assembled immunoglobulin protein.

“Total protein” and “total soluble protein” are used interchangeably, unless otherwise specified. Thus, unless otherwise noted, any given weight of total protein measured should be interpreted by the skilled artisan to mean total soluble protein. Further, a value given in μg/mg TSP to the corresponding value given in % TSP. As an example, 1 μg/l mg TSP is equivalent to 1 μg per 1000 μg TSP (or 0.001 μg/μg TSP) which, expressed as a percentage of TSP in μg weight, would be 0.1% TSP measured in μg. For example, 30.3 μg/mg total (soluble) protein. This translates to 0.0303 μg per μg TSP, which, stated as a percentage, equals 3.03% TSP.

Units can also be expressed as μg per grain of monocot seed. This weight can be correlated with the percentage of total soluble protein, given that the average weight of a seed/grain and how much of this weight is represented by the TSP are matters readily known to skilled artisans. For example, the 1000 grain weight of rice is, on average, approximately 20-25 grams, which translates to 20-25 mg (or 20,000-25,000 μg) per rice grain. As one example, a transgenic rice plant may typically yield 190 μg total soluble protein per grain which is roughly equivalent to 0.8% grain weight (190 μg divided by 25,000 μg=0.0076 which is rounded up to 0.8%).

As is known in the art, “endosperm” or “endosperm tissue” is a seed storage tissue found in mature seeds.

The terms “crude extract,” “partially-purified” or “substantially unpurified” means that a composition made from the transgenic monocot seed is not subjected to significant purification steps, such as chromatographic protein purification and fractionation steps.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Proof of Concept—Expression of Secretory IgA in Rice Grains

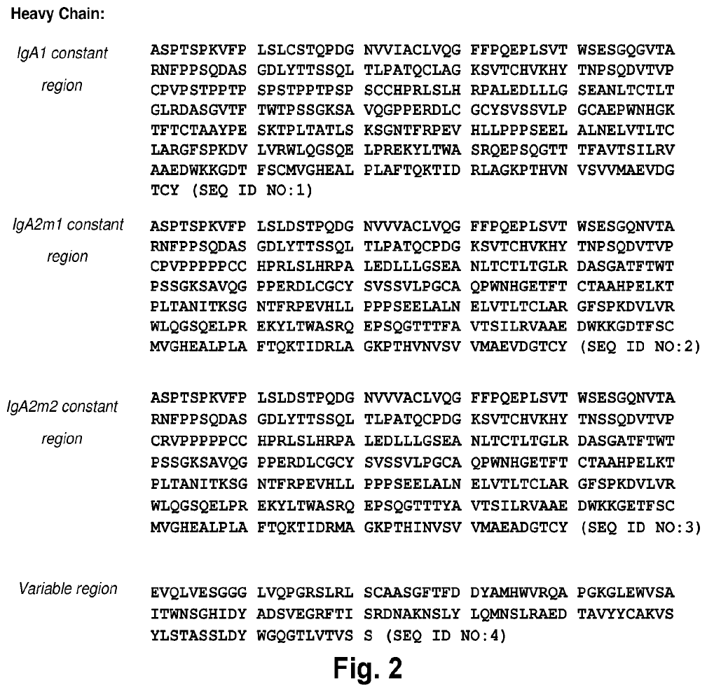

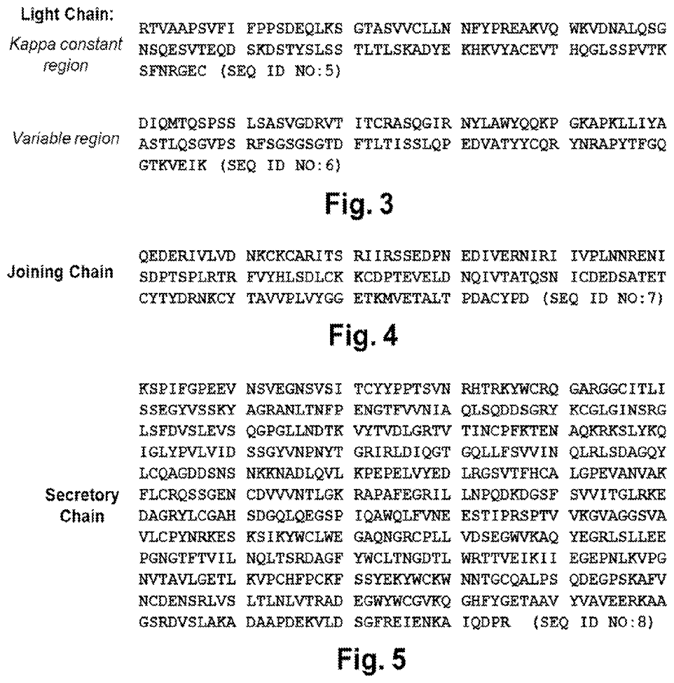

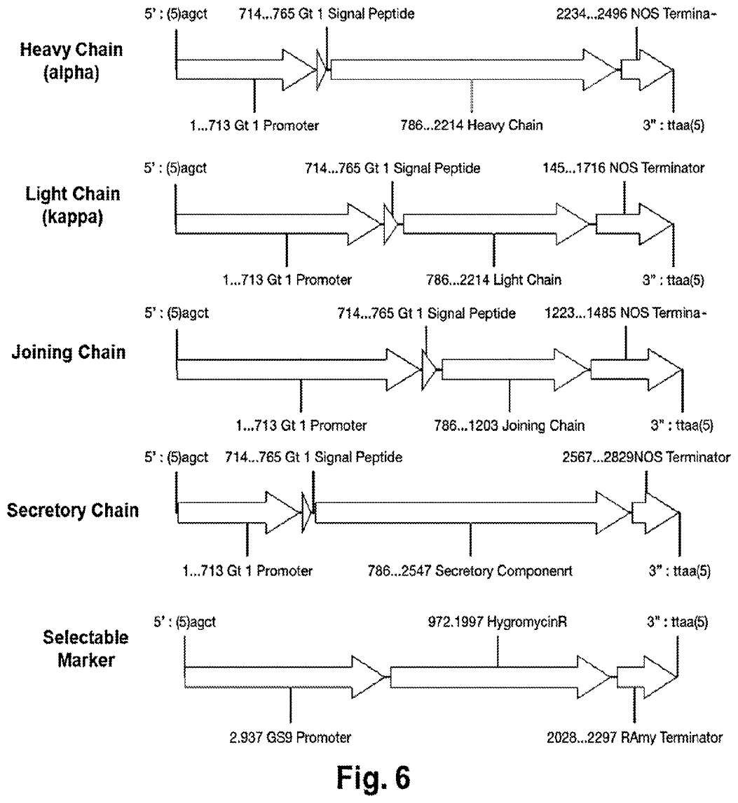

Development of Gene constructs—To obtain high expression levels of recombinant SIgA in rice grains, four gene constructs were developed for sIgA's LC, HC, J chain, and secretory component, respectively (FIGS. 2-5). Each of the above four protein amino acid sequences was back-translated into a nucleotide sequence with the codons optimized towards the codon-usage preference of the host expression system, 0. Sativa, while the internal repeats and other features that might affect mRNA stability or translation efficiency were altered. The entire nucleotide sequence was synthesized, and then ligated in frame into a backbone plasmid vector called pAPI405, which contains rice seed storage protein gene (GenBank accession no. Y00687) promoter (Gt1) SEQ ID NO:16, signal peptide encoding sequence, and the terminator of the nopaline synthase (nos) gene of the T-DNA in Agrobacterium tumefaciens (SEQ ID NO: 17) (FIG. 6). Thus, in each expression vector, the sIgA component optimized coding sequence is operably linked to the downstream of rice seed storage protein glutelin 1 gene promoter (Gt1) including its signal peptide encoding sequence (GenBank accession no. Y00687) and to the upstream of the nopaline synthase (nos) gene terminator. In the case of heavy chain and light chain, the respective variable and constant regions are linked in series and inserted between the promoter and terminator:

Heavy chain: Promoter—Variable-Constant—Terminator (SEQ ID NO:19)

Light chain: Promoter—Variable-Constant—Terminator (SEQ ID NO:20)

Joining chain: Promoter—Joining Chain—Terminator (SEQ ID NO:21)

Secretory component: Promoter—Secretory Component—Terminator (SEQ ID NO:22) The resulting plasmids were verified by sequencing in both orientations, and designated as pVB85 for the expression of the heavy chain (SEQ ID NO:19), pVB86 for the expression of the light chain (SEQ ID NO:20), VB87 for the expression of the J chain (SEQ ID NO:21), and pVB88 for the expression of the secretory component (SEQ ID NO:22).

The plasmid pAPI146 was used to provide a selection marker in plant transformation. The pAPI146 consists of the hpt (hygromycin B phosphor-transferase) gene encoding the hygromycin B-resistant protein under the control of rice beta-glucanase 9 gene promoter, which restricts the expression of hpt gene only in rice calli

Plant genetic transformation—The linear expression cassettes of DNA fragments comprising the region from promoter to terminator (without the backbone plasmid sequence) in VB85, VB86, V87, and VB88 plasmid vectors were released with EcoRI and HindIII double digestion and used for microprojectile bombardment-mediated co-transformation of embryonic calli induced from the mature seeds (Oryza sativa, subsp. Japonica).

| | |
|---|---|
| Rounds of Bombardment | 15 |
| Calli Bombarded | 11,023 |
| Events Generated | 354 |
| High Expressing Events >.5 g/kg | 8 |
| Med Expressing Events >.1 g/kg | 56 |
| Low Expressing Events >.05 g/kg | 71 |
| Sterile, Dwarfed, Dead | 219 |

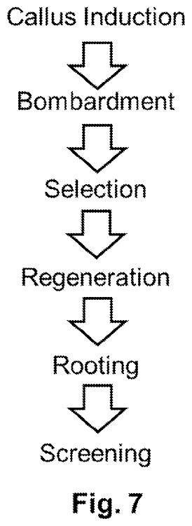

Transgenic rice plants containing the four transgenes encoding sIgA's four components, i.e., LC, HC, J chain, and secretory components, were then identified by PCR using primers specific to the nucleotides of the four genes, and then transferred to soil to be grown in a greenhouse (FIG. 7). The regenerated transgenic plants are referred to as R0 plants or transgenic events, and their progeny in successive generations are designated as R1, R2, etc.

Expression screening analysis of transgenic seeds—Out of 354 transgenic events (R0), 135 were harvested (R1) for expression analysis. To identify transgenic plants that express the full sIgA, multiple seeds were analyzed due to the genetic segregation of hemizygous transgenes in the selfed R1 seeds. Eight R1 seeds from each transgenic event were randomly picked, dehusked, and placed into eight wells in the same column of a 96-well 1 ml of microplate. Two hundred microliters of PBS buffer (pH 8.3) and two 4-mm diameter steel beads were dispensed into each well. Then homogeneous seed protein extracts were produced by agitating the plate with a Geno/Grinder (SPEX, NJ) for 10 min at 1,300 strokes/min followed by centrifugation with a microplate centrifuge at 4,000 rpm for 20 min. Equal amount of supernatant protein extracts from each seed were pooled. Three microliters of the pooled crude protein extract from each transgenic event were spotted onto a nitrocellulose membrane. The blot was blocked in 5% non-fat milk in tris buffered saline tween-20 (TBST) for 1 hour, and then incubated with one of the four antibodies: anti-light chain antibody, anti-heavy chain antibody, anti joining chain antibody, and anti-secretory component antibody for 1 hour. After being washed four times, five minutes each with TBST buffer, the dot blots were incubated with a corresponding HRP (horseradish peroxidase)-conjugated antibody in TBST buffer for 1 hour followed by four washes in TBST buffer, 5 min each. Then, the blots were incubated with chemiluminescence substrate (SuperSignal, MA) for five minutes, and the immune reaction signals were detected with Protein Simple Imager. In total, 68 positive transgenic events expressing the full sIgA were identified by immuno dot-blot expression analysis.

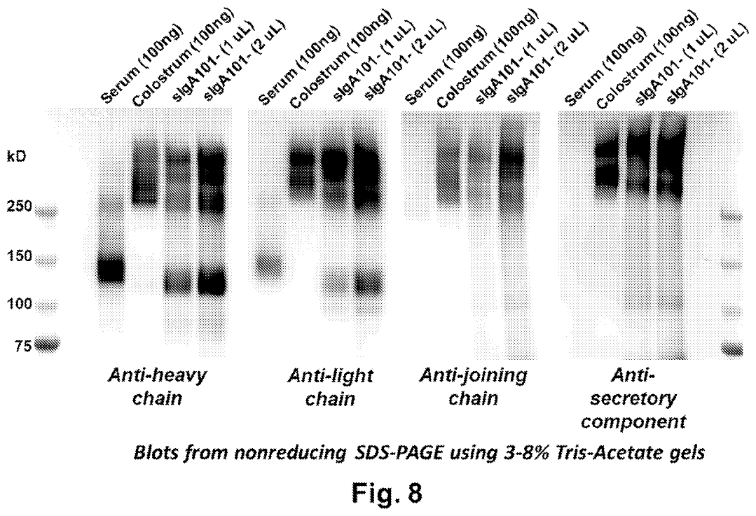

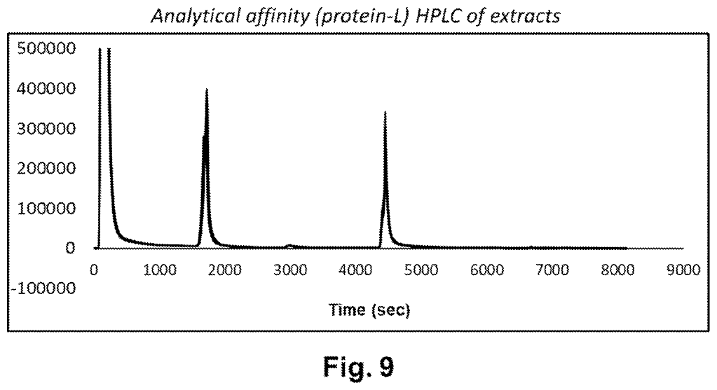

The Western blot analysis further demonstrated that the recombinant sIgA cross-reacted specifically with each anti-sIgA individual component (heavy chain, light chain, joining chain and secretory component) antibody under reduced conditions as well as denatured condition. Furthermore, an immune Western blot under the native condition showed that all the four components of rice-produced recombinant sIgA were fully assembled into a multi-complex antibody with the same molecule size as human colostrum-derived sIgA (FIG. 8).

The selection of homozygous lines—To select the homozygous lines expressing rsIgA, R1 seeds of each selected transgenic rice event were grown to the next generation. For each R1 line, over 20 R2 seeds were assayed by immuno-dot-blot to evaluate the genetic segregation of rsIgA expression. The immune dot blot protocol was the same as described above. The lines with all 20 R2 seeds shown as positive were considered homozygous.

Functional characterization of rice produced rsIgA—To assess whether rice-derived sIgA is bioactive (potent), the rsIgA was evaluated for its ability to bind its targeting antigen, human tumor necrosis factor alpha (TNF-α). A sandwich-type ELISA assay was performed for this evaluation. Briefly, an ELISA microplate was coated with TNF-α in a 100 mM bicarbonate/carbonate buffer. Then, different amount of protein extracts from rice seeds expressing sIgA were added into the wells of TNF-α—coated microplate and inoculated for one hour. After washing in TBST buffer four times, five minutes each, HRP-conjugated anti-human secretory component antibody was added to microplate wells. After a 1-hour incubation at room temperature, the plate was washed four times with TBST buffer, 5 minutes each. The plate was then developed using TMB substrate and absorbance readings at OD450 were recorded.

Quantification of rice derived sIgA—To determine expression levels of the rice derived sIgA antibodies, a sandwich ELISA was performed. An ELISA plate was coated using 500 of a 2 ug/ml solution of anti-human IgA antibody with 100 mM bicarbonate/carbonate buffer. After an overnight incubation, the plate was washed with TBS-T. A blocking buffer containing 1×BSA was then added and incubated for 2 hours at room temperature. The plate was then washed twice with TBS-T. Positive controls and crude rice extracts were made into several serial dilutions and added to the plate. After shaking for 2 hours at room temperature, the plate was washed 4 times with TBS-T. HRP conjugated anti-human heavy chain antibodies were then added. After a 1-hour incubation at room temperature, the plate was washed 4 times with TBS-T. The plate was then developed using TMB substrate and absorbance readings at OD450 were recorded. A standard curve was created and crude extracts were compared and quantified.

Purification of recombinant sIgA from rice grains—In order to produce a purified rice derived sIgA antibody, 2 grams of milled rice flour was added to 20 ml of extraction buffer containing 200 mM Tris, 150 mM Sodium Chloride, 5 mM EDTA, 0.1% Tween20, and 0.00% Sodium Azide, final pH 8.8. The sample was extracted for 30 minutes on an orbital shaker. The sample was then spun down using centrifugation at 4,000×g for 20 minutes. The supernatant was filtered using a 0.2 μm filter and the buffer was exchanged using a 50K diafiltration membrane. The final buffer solution was TBS at pH 8.5. For this purification, a 1 ml Protein L prepacked Hi-Trap column (GE, CT) was used. The column was equilibrated with 5 column volume (cv) of binding buffer. The sample was then loaded at a rate of 1 ml per minute and washed with 5 cv of binding buffer. The sample was then eluted using a Sodium citrate buffer (pH 2.0) into ten 500 μl fractions. Each fraction contains 500 μl of neutralization buffer, pH 12, to offset the low pH of the elution buffer. The column was then re-equilibrated, cleaned, and stored in a 20% ethanol solution. Three μl of each fraction, precolumn samples, flow through, and wash were placed on nitrocellulose and probed using anti-heavy chain antibodies. Results show the majority of the sIgA antibodies eluted out in the first 2 to 3 ml.

### Example 2

Work was carried out to confirm the inclusion and assembly of all expressed chain components of sIgA and verify high-level expression of assembled sIgA and IgA in seed extracts of a rice transformant. Nonreducing SDS-PAGE followed by western blotting of milled rice flour extract with antibodies specific for each sIgA component shows that all four chain types are present in high-molecular weight species that migrate similarly to commercial samples of purified sIgA from human colostrum and purified IgA from human plasma serum.

Protocol: Selected amounts of milled flour from sIgA101-expressing rice were extracted by agitation in defined extraction buffer (e.g. 150 mM NaCl, 200 mM Tris-HCl pH 8.3, 0.1% Tween-20, 5 mM EDTA) at 10:1 volume:weight ratio. The spent flour was separated from the extract by low-speed centrifugation followed by rapid filtration of the supernatant using a syringe filter (e.g. Millipore 0.2 or 0.45 μm, 25 mm diameter cellulose acetate disc filter). The clarified extract, along with monomeric IgA and sIgA reference standards (purified commercial serum IgA and colostrum IgA preparations) were subjected to nonreducing SDS-PAGE on low-percentage Tris-Acetate minigels (e.g. Invitrogen NuPage 3-8% Tris-Acetate gels) to maximize the separation of high-molecular weight species in the 100-500 kDa range. In a typical protocol, the samples were separated at 180 volts for 90 minutes.

Following SDS-PAGE, samples were electrotransferred to PVDF membranes in Tris-Glycine-SDS transfer buffer containing 20% v/v Methanol. A typical transfer was carried out at 100 volts for 60 minutes. Transfer efficiency was confirmed by staining the gels (post-transfer) with Coomassie R-250-based (e.g. Pierce Gelcode Blue) or SYPRO-Ruby fluorescent protein stains to detect untransferred protein. The PVDF membranes were blocked for 60-90 min. with 5% w/v milk powder in 1×TBS-T (Tris-Buffered saline+0.05% v/v Tween-20). Following blocking, the membranes were washed 3-4 times for at least 5 mins per wash in TBS-T and incubated for 2-12 hours with sIgA chain-specific antibody-HRP conjugates (anti-HC, -LC, or- SC) or primary antibodies (anti-JC). Membranes were rewashed 3-4 times with TBS-T for at least 15 mins per wash. The anti-JC blot was reprobed for 30-60 minutes with a specific secondary antibody-HRP conjugate, followed by rewashing 3-4 times for at least 15 mins per wash. Detection of bound antibodies was carried out using commercial chemiluminescent developing reagents (e.g. ThermoFisher Super Signal West) and imaged using a CCD camera-equipped imager (ProteinSimple Fluorchem Q imager). The results are shown in FIG. 8.

Next, rice plant extracts were analyzed. Peak-A is flow-through of unretained material loaded in mobile phase-A; Peak-B represents elution of non-specifically bound proteins with mobile phase-B; Peak-C shows the elution of specific protein-L-bound target proteins (e.g. sIgA) eluted with mobile phase-C. Measurement of the area under Peak-C allows quantification of the high expression level of IgA species obtainable using the ExpressTec system. Chromatography conditions: column size is 1 mL, 10 mm×50 mm. Resin is TOYOPEARL AF-rProtein L-650F from TOSOH. Mobile phase-A: 10 mM sodium phosphate pH 7.0; mobile phase-B: 10 mM sodium phosphate, 500 mM sodium chloride pH 7.0; mobile phase-C: 20 mM sodium phosphate pH 2.0.

Protocol: 100 mg of sIgA101 flour was resuspended in an extraction buffer containing 150 mM NaCl, 200 mM TRIS-HCl pH 8.3 and homogenized using a glass Dounce homogenizer with 50 strokes. The soluble portion (supernatant) of the homogenized flour suspension was separated from solids by centrifugation, decanted and concentrated using ultracentrifugal concentrators (e.g. Amicon/Microcon 10K NMWL from Millipore). The extract was concentrated to 100 μL and diafiltered against 4 volumes of the mobile phase-A (10 mM sodium phosphate pH 7.0) to complete buffer exchange. The volume of the final sample was adjusted to 200 and the sample was injected on an analytical protein-L HPLC column (e.g. TOSOH TOYOPEARL AF-rProtein L-650F). Nonspecifically bound proteins were eluted in a high salt buffer, mobile-phase B (10 mM sodium phosphate, 500 mM sodium chloride, pH 7.0). Kappa-light-chain-containing protein complexes that bound specifically to protein-L were eluted in an acidic buffer, mobile phase-C (20 mM Sodium phosphate 2.0). The sIgA content in the extract was quantified by referencing the area under the peak obtained by elution with mobile phase-C to calibration curves obtained from sIgA reference standards of known amounts. The results are summarized in the table below and shown in FIG. 9.

TABLE
Quantitation of Total sIgA protein from sIgA101 (Line B) extraction

| | Volume of | Amount | Amount (mg) | Avg amount | |
|---|---|---|---|---|---|
| Concentration | injection | (mg) in | per gram | (mg) per gram | |
| (mg/mL) | (mL) | injection | of rice flour | rice flour | Std |
| 1.38 | 0.2 | 0.277 | 2.15 | 2.63 | 0.427 |
| 1.87 | 0.2 | 0.375 | 2.97 | | |
| 1.76 | 0.2 | 0.353 | 2.78 | | |

Next, chromatographic separation of protein-L purified sIgA from IgA species was carried out using preparative gel filtration chromatography (25 mL Superose 6 resin in a 10 mm diameter column, 30 mm length). Protein-L purified total IgA mixtures are well-resolved into sIgA and IgA. Trace aggregates and lower-molecular weight contaminants, including partially- or incorrectly-assembled IgA-like species) are also resolved. The major peaks are analyzed using nonreducing and reducing SDS-PAGE and compared to commercial samples of purified sIgA from human colostrum and purified IgA from human plasma serum. The analysis reports on the identity and relative purity of each peak, verifies the presence of the expected component chains, and confirms the proper assembly states.

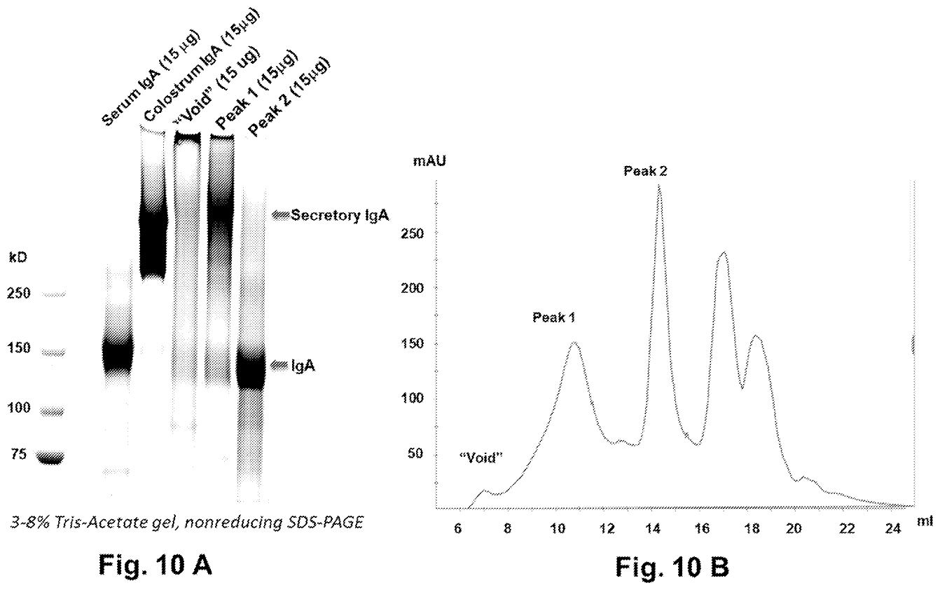

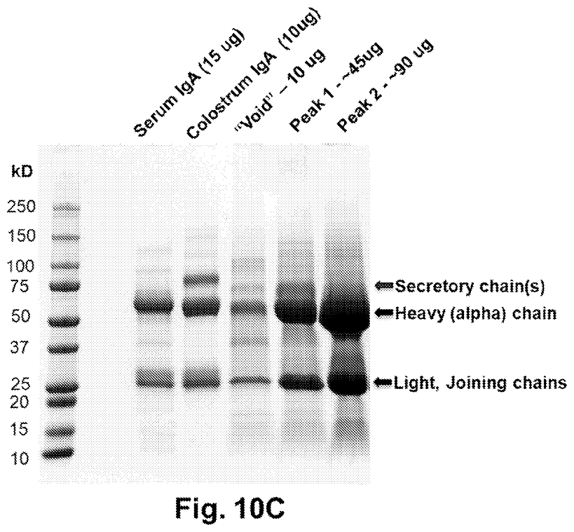

Protocol: Protein-L purified sIgA peak was concentrated to the appropriate volume by centrifugal concentration using, e.g., Millipore Amicon/Microcon Cellulose Acetate concentrators. The concentrated volume was selected to be 2-5% of the bed volume of a gel filtration column (e.g. <0.5 mL for a 25 mL Superose 6 column). The concentrated sample was loaded manually at low flow rate onto the column. The column was eluted with isocratic flow of gel filtration buffer (e.g. 150 mM NaCl, 50 mM Tris-HCl pH 8.5, 0.1% Tween-20) at low flow rates (<0.5 mL/min) to maximize resolution. Peak fractions were pooled and analyzed by nonreducing and reducing SDS-PAGE and total protein staining (Coomassie R250, e.g. Gelcode Blue stain, or SYPRO-Ruby fluorescent protein stain). The results are shown in FIG. 10A-10C.

Next, the identity of sIgA and IgA in protein-L purified, size-fractionated IgA samples (as shown in FIG. 10A-C) was confirmed by Western blotting. Immunoblots confirm the inclusion and assembly of all expressed chain components of sIgA, showing that the purified fractions contain the correct chain types in high-molecular weight species that migrate similarly to commercial samples of purified sIgA from human colostrum and purified IgA from human plasma serum.

Protocol: Pooled peaks from preparative gel filtration of protein-L purified sIgA species were subjected to nonreducing SDS-PAGE on low-percentage Tris-Acetate minigels (e.g. Invitrogen NuPage 3-8% Tris-Acetate gels) to maximize the separation of high-molecular weight species in the 100-500 kDa range. In a typical protocol, the samples were separated at 180 volts for 90 minutes.

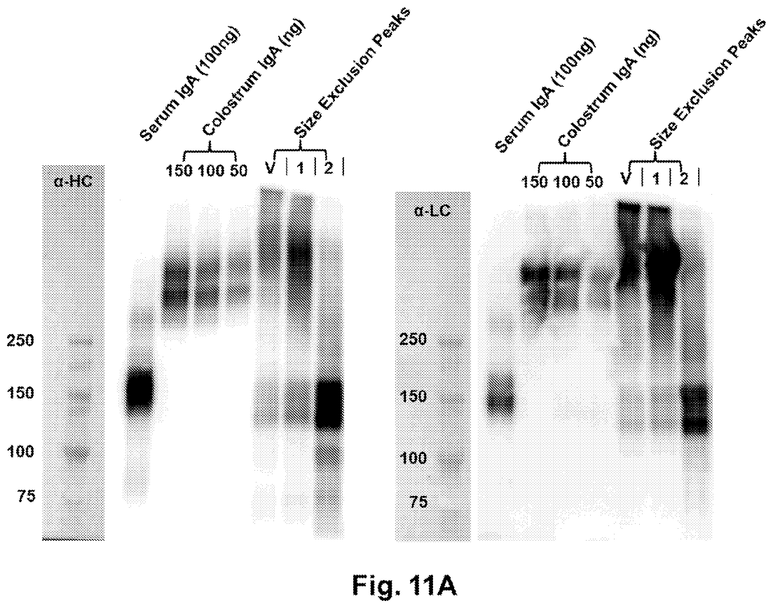

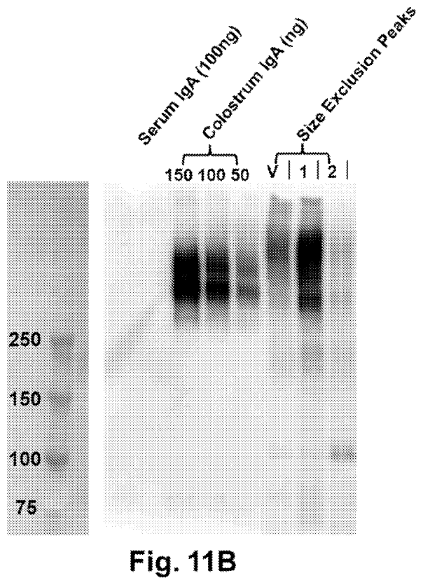

Following SDS-PAGE, samples were electrotransferred to PVDF membranes in Tris-Glycine-SDS transfer buffer containing 20% v/v Methanol. A typical transfer was carried out at 100 volts for 60 minutes. Transfer efficiency was confirmed by staining the gels (post-transfer) with Coomassie R-250-based (e.g. Pierce Gelcode Blue) or SYPRO-Ruby fluorescent protein stains to detect untransferred protein. The PVDF membranes were blocked for 60-90 min. with 5% w/v milk powder in 1×TBS-T (Tris-Buffered saline+0.05% v/v Tween-20). Following blocking, the membranes were washed 3-4 times for at least 5 mins per wash in TBS-T and incubated for 2-12 hours with sIgA chain-specific antibody-HRP conjugates. Membranes were rewashed 3-4 times with TBS-T for at least 15 mins per wash. Detection of bound antibody conjugates was carried out using commercial chemiluminescent developing reagents (e.g. ThermoFisher Super Signal West) and imaged using a CCD camera-equipped imager (ProteinSimple Fluorchem Q imager). The results are shown in FIG. 11A-11B. Further work has been carried out using IgA sequences in the Sequence Listing.

### Example 3

### In Vitro Study: L929

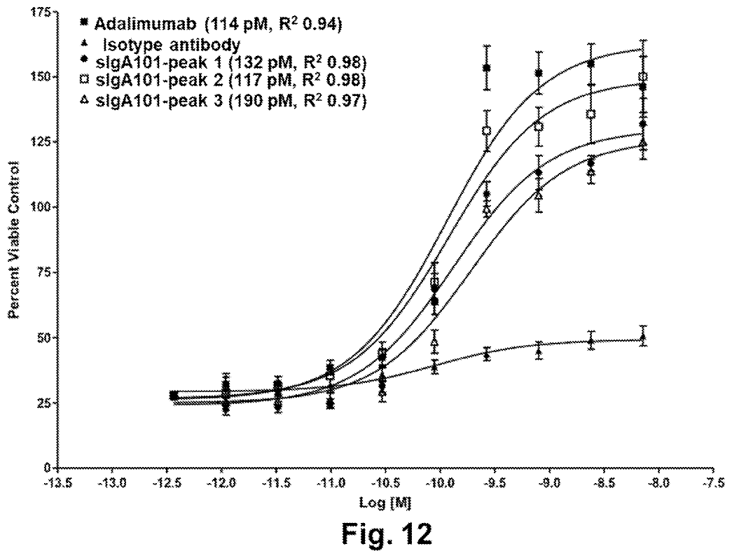

Murine fibrosarcoma L929 cells (ATCC, Manassas, Va.) were cultured in L929 growth media (EMEM supplemented 2 mM Glutamine and 10% horse serum (Sigma Aldrich, St. Louis, Mo.) and 1× antibiotics (Life Technologies, Grand Island, N.Y.)). Cells were plated at an initial cell density of 10,000 cells/well in flat-bottom tissue culture-treated 96-well plates in L929 growth media and allowed to adhere to the plate for at least 3 hours at 37° C. Adalimumab (Abbvie, Chicago, Ill.), colostrum IgA isotype control (Sigma Aldrich, St. Louis, Mo.), or SIgA101 (Ventria Bioscience, Junction City, Kans.) were diluted to a top concentration of 28.4 nM, and serial 3-fold dilutions were generated in a separate 96-well plate in L929 growth media. Antibody was added to the L929 cells as a 4-fold concentrate. Human TNF (Peprotech, Rocky Hill, N.J.) was added to the L929 cells to a final concentration of 5 ng/mL. Cells were incubated at 37° C. 5% CO₂ for 72 hours. At the end of the 72 hours, 2.5 μL of MTT reagent (EMD Millipore, Billerica, Mass.) diluted in 7.5 μL in L929 growth media per well of cells. Cells were incubated in the presence of the MTT reagent for 4 hours at 37° C. 5% CO₂. Isopropanol supplemented with 0.4N HCl was used to lyse the L929 cells. Viability of cells was determined using a wavelength 570 nm with a correction wavelength of 630 nm. Each concentration point for each drug treatment was run in duplicate per plate on triplicate plates. Each concentration point was normalized to the TNF-untreated controls (n of 8 per plate). The results are shown in FIG. 12. Data was fit to a nonlinear regression using a sigmoidal dose-response model and EC50s were calculated (GraphPad Prism). Data presented is the mean of 4 independent experiments. EC50s have been averaged between the 4 experiments.

### In Vitro Study: TEER

Determination of Antibody-Mediated Transepithelial Resistance Protection in TNF-Induced T84 monolayer permeability.

Human intestinal epithelial cells (T84) were grown and maintained in T75 cell culture flasks (Costar, Cambridge Mass.) in DMEM/F12 (Ham) medium (Mediatech, Manassas, Va.) containing 5% Fetal Bovine Serum (FBS; Mediatech, Manassas, Va.). cells were cultured at 37° C. in a humidified atmosphere with 5% (v/v) CO₂. Medium was replenished every 3-4 days.

The cells were seeded in 24-well Transwell chambers (Millicell PET; 0.4 μM pore size; Millipore) at a density of 8.0×104/cm2. TEER were monitored using an EVOM voltohmmeter (World Precision Instruments, Sarasota, Fla.) until the resistances of the monolayers reached high resistance (<2,000 Ω·cm2), during which time, basolateral media were changed every 3-4 days. Once monolayers reached high resistance (>21 days), media was removed and replaced with fresh DMEM/F12+5% FBS on the apical compartment and DMEM/F12+5% FBS containing 10 ng/mL recombinant interferon-γ (IFN-γ; Peprotech, Rocky Hill, N.J.) and incubated overnight, as previously described.

A 10× concentrated cocktail of tumor necrosis factor-α (TNF-α; 50 ng/mL in DMEM/F12 +5% FBS) was prepared with 10× each separate study material (antibody): human secretory immunoglobulin A from human colostrum (sIgA; Sigma Aldrich, St. Louis, Mo.), adalimumab (recombinant anti-human TNFα antibody, clone D2E7; Cedarlane, Burlington, N.C.), rice derived recombinant anti-human TNFα (SIgA101; Ventria Bioscience, Fort Collins, Colo.). The 10× concentrate of antibodies ranged from 0.03-0.01 nM.

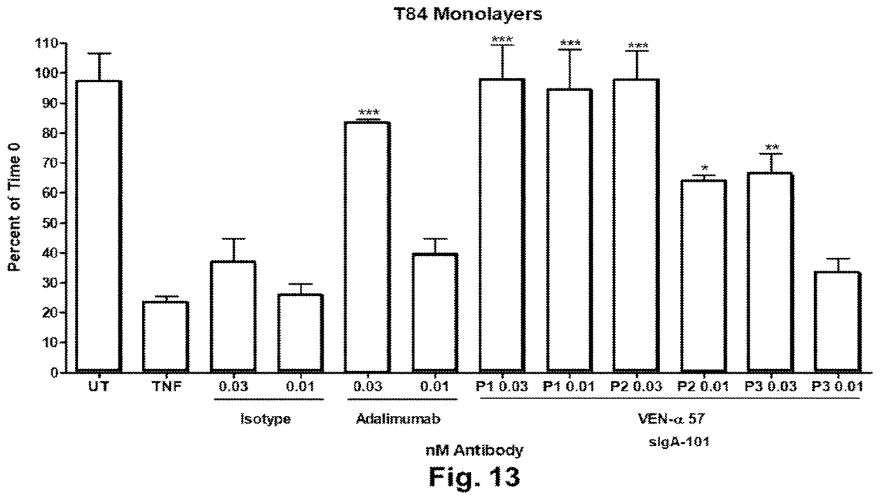

Prior to incubation with TNF and antibody, TEERS were obtained for each monolayer. The basolateral media were then spiked with the 10×cocktail to a final concentration of 5 ng/mL TNFα and 0.3-0.01 nM each antibody. Cells were incubated for 16 h and TEER measurements obtained. TEER were expressed as a percentage of the control sample (containing INFγ, with no TNF or antibody) and analyzed using Prism software (Graphpad, La Jolla, Calif.). The results are shown in FIG. 13. Data presented is the mean response of T84 cells in 2 independent experiments. *** p≤0.001 by One Way ANOVA.

### In Vivo Study: DSS Pilot Study

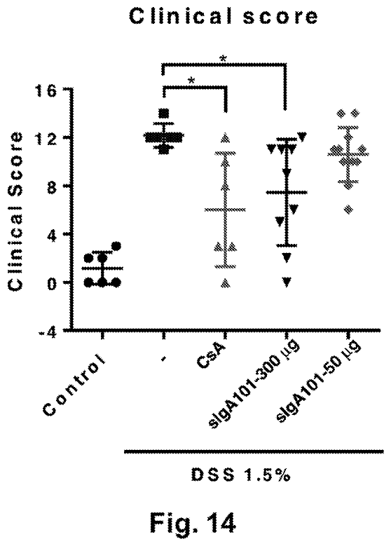

6-8 week old C57/BL6 mice (Jackson Laboratories, Bar Harbor, Me., USA) were allowed to acclimate to the study site's vivarium for one week. At day 0, mice were treated with dextran sodium sulfate (DSS) ad libitum (1.25% w/v 36-50 kDa, #0216011050 MP Biomedicals, Santa Ana, Calif., USA) in drinking water for 6 d. Fresh DSS solution was replaced mid-way through the study, at D3. Also starting at day 0, DSS groups received daily gavage of Cyclosporin (#C3662 Sigma Aldrich 50 mg/kg via oral gavage, 200 μL volume), VEN-alpha (50 μg-300 μg/day, 100 μl gavage volume) at the same time each day, or sham (mock buffer, Ventria Bioscience). Oral treatments (test article and oral controls) continued daily until the termination of the experiment at d7. Overall clinical score was evaluated by the percentage of weight loss from the initial body weight (measured daily), colon length reduction at day 7, and the presence of intestinal bleeding (measured daily and cumulative score determined). Each parameter was totaled to generate a cumulative clinical score that is presented. The results are summarized in FIG. 14 and in the table below.

TABLE
In vivo model

| | | | | DSS + | DSS + |
|---|---|---|---|---|---|
| Clinical | | | DSS + | sIgA101 | sIgA1010 |
| parameters | Control | DSS | CsA | (300 μg) | (50 μg) |
| Change in body | 0.7 + 0.12 | −3.3 + 0.20 | −1.1 + 0.63 | −1.3 + 0.54 | −2.4 + 0.41 |
| weight (g/7 days) | | | | | |
| Colon length | 74.5 + 2.99 | 56.3 + 1.24 | 62.1 + 3.71 | 60.9 + 2.69 | 54.1 + 1.32 |
| (mm) | | | | | |
| Cumulative | 0 | 8.3 + 1.67 | 1.3 + 0.84 | 3.6 + 1.44 | 7.0 + 1.36 |
| bleeding score | | | | | |

Mean + SEM;
n = 6 to 12 mice per group

### Example 4

### Proof of Concept—Expression of Secretory IgA in Barley Grains

Development of Gene constructs—To obtain high expression levels of recombinant SIgA in barley grains, four gene constructs are developed for sIgA's LC, HC, J chain, and secretory component, respectively, as described in Example 1 above. Each of the four protein amino acid sequences are back-translated into a nucleotide sequence with the codons optimized towards the codon-usage preference of the host expression system, Hordeum sativa, while the internal repeats and other features that might affect mRNA stability or translation efficiency are altered. Subsequent DNA transformations are otherwise identical to that demonstrated in Example 1 for rice.

Plant genetic transformation—The linear expression cassettes of DNA fragments comprising the region from promoter to terminator (without the backbone plasmid sequence) in VB85, VB86, V87, and VB88 plasmid vectors are released with EcoRI and HindIII double digestion and used for microprojectile bombardment-mediated co-transformation of embryonic calli induced from the mature seeds (H. sativa, Golden Promise), as developed by Wan and Lemauz, using microparticle bombardment. Generation of Large Numbers of Independently Transformed Fertile Barley Plants, Plant Physol. Vol. 104, 1994

### Plant Materials

Plants of the barley (Hordeum vulgare L.) spring cultivar Golden Promise are grown in growth chambers under a 16-h light/8-h dark period at 12° C. and 60 to 80% humidity. Light levels at head height are approximately 350 to 400 μE. When about 10 cm in height, the seedlings are vernalized for 8 weeks under a 10-h light (10-15 μE)/14-h dark period at 4° C. After vernalization, they are returned to the original growing conditions. Plants receive Osmocote (Sierra, 17-6-12 plus minors), then biweekly with Peter's 20-20-20.

Callus Derived from Immature Embryos

Spikes with immature embryos (1.5 to 2.5 mm) are surface sterilized for 5 min in 20% (v/v) bleach, rinsed briefly three times, and washed for 5 min with sterile water. Immature embryos are dissected from young caryopses and left intact or are bisected longitudinally. For induction of callus for bombardment, embryos (intact or bisected) are placed scutellum-side down on callus induction medium consisting of Murashige and Skoog medium supplemented with 30 g/L maltose, 1.0 mg/L thiamine-HCl, 0.25 g/L myo-inositol, 1.0 g/L casein hydrolysate, 0.69 g/L Pro, and 2.5 mg/L dicamba, solidified by 3.5 g/L Gelrite. Embryos are incubated at 25° C. in the dark, and embryogenic callus was selected for bombardment after 2 weeks.

### Microprojectile Bombardment

Approximately 0.5 g of embryogenic callus is cut into 2 mm pieces and placed in the center of a Petri dish (100×15 mm) containing callus induction medium. Purified DNA fragments encoding the gene of interest and hygromycin B phosphotransferase gene are adsorbed to gold particles and bombarded once with a DuPont PDS 1000 He Biolistic Delivery System. The target materials are positioned approximately 13 cm below the microprojectile stopping plate; 1100-p.s.i. rupture discs are used.

### Selection of Transformants

One day after bombardment, callus pieces are transferred individually to callus induction medium with hygromycin B. Tissue remains on selection 10 to 14 d. At transfer to the second selection plate, callus pieces are broken into several small pieces and maintained separately. During the subsequent selection passages callus pieces showing evidence of more vigorous growth are transferred earlier to new selection plates and tissue is handled in an identical manner.

### Regeneration

Plants are regenerated from HygB resistant callus lines by transferring embryogenic callus to shooting medium with at 23° C. under fluorescent lights (45-55 μE, 16 h/d). In approximately 2 weeks, plantlets are observed. Green plantlets, approximately 2 cm are transferred into Magenta boxes containing plantlet growth medium (hormone-free callus induction medium. Before they grow to the top of the box, plantlets are transferred to 6-inch pots containing Supersoil and placed in the greenhouse (16-h light period, 18° C. Regenerants grow to maturity and are self-pollinated.

Transgenic barley plants containing the four transgenes encoding sIgA's four components, i.e., LC, HC, J chain, and secretory components, will be identified by PCR using primers specific to the nucleotides of the above four genes (FIGS. 2-5), and then transferred to soil to be grown in a greenhouse. The regenerated transgenic plants are referred to as R0 plants or transgenic events, and their progeny in successive generations are designated as R1, R2, etc.

Expression screening analysis of transgenic seeds—This is done in the same manner as with rice.

The selection of homozygous lines—To select the homozygous lines expressing rsIgA, R1 seeds of each selected transgenic barley event are grown to the next generation. For each R1 line, over 20 R2 seeds are assayed by immuno-dot-blot to evaluate the genetic segregation of rsIgA expression. The immune dot blot protocol is the same as described above. The lines with all 20 R2 seeds shown as positive are considered homozygous.

Functional characterization of barley-produced rsIgA—To assess whether barley-derived sIgA is bioactive (potent), the rsIgA is evaluated for its ability to bind its targeting antigen, human tumor necrosis factor alpha (TNF-α). This is accomplished via a sandwich-type ELISA assay as with rice.

Quantification of barley derived sIgA—To determine expression levels of the barley derived sIgA antibodies, a sandwich ELISA is performed as with rice.

Purification of recombinant sIgA from barley grains—In order to produce a purified barley-derived sIgA antibody, 2 grams of milled barley flour is added to 20 ml of extraction buffer containing 200 mM Tris, 150 mM Sodium Chloride, 5 mM EDTA, 0.1% Tween20, and 0.00% Sodium Azide, final pH 8.8. The sample is extracted for 30 minutes on an orbital shaker. The sample is then spun down using centrifugation at 4,000×g for 20 minutes. The supernatant is filtered using a 0.2 μm filter and the buffer is exchanged using a 50K defiltration membrane. The final buffer solution is TBS at pH 8.5. For this purification, a 1 ml Protein L prepacked Hi-Trap column (GE, CT) is used. The column is equilibrated with 5 column volume (cv) of binding buffer. The sample is then loaded at a rate of 1 ml per minute and washed with 5 ml of binding buffer. The sample is then eluted using a Sodium citrate buffer (pH 2.0) into ten 500 μl fractions. Each fraction contains 500 μl of neutralization buffer, pH 12, to offset the low pH of the elution buffer. The column is then re-equilibrated, cleaned, and stored in a 20% ethanol solution. Three μ.1 of each fraction, precolumn samples, flow through, and are placed on nitrocellulose and probed using anti-heavy chain antibodies. Results show the majority of the sIgA antibodies elute out in the first 2 to 3 ml.

### Exemplary Immunoglobulin Sequences

As noted, the present approach may be extended to other immunoglobulins. Examples include the following, which are incorporated by reference herein.

| Category/ | | | Accession Number | |
|---|---|---|---|---|
| Indication | Target | Name | (Database) | Reference |
| Autoimmune | TNF-alpha | Infliximab | 5VH3 and 5VH5 | |
| | | | (PDB) | |
| | Integrin | Vedolizumab | DB09033 | |
| | Alpha4/Beta7 | | DB05802 | |
| | Integrin Alpha4 | Natalizumab | | |
| | Beta 7 Integrin | Etrolizumab | DB12189 | |
| | | | (Drugbank); D09901 | |
| | | | (KEGG DRUG) | |
| | CD20 | Rituximab | DB00073 (BTD00014, | |
| | | | BIOD00014) | |
| | EGFR | Cetuximab | DB00002 (BTD00071, | |
| | | | BIOD00071) | |
| | EGFR | Panitumumab | 5SX4 (PDB) | |
| | IL-12/IL-23 | Ustekinumab | | |
| | IL-23 | Guselkumab | DB11834 | |
| | IL-6 | Siltuximab | DB09036 | |
| | IL-6 receptor | Tocilizumab | DB06273 | U.S. Pat. No. 8,398,980 |
| | | | (Drugbank) | |
| | IL-17 | Secukinumab | DB09029 | |
| | IL-17 | Ixekizumab | DB11569 | |
| | CD3 | UCHT1 | | |
| | IL-1beta | Canakinumab | DB06168 | |
| | | | (Drugbank) | |
| Infectious | Respiratory | Palivizumab | DB00110 | U.S. Pat. No. 6,988,717 |
| Disease | syncitial virus | | (Drugbank) | US20030097974 |
| | glycoprotein F | | | US20060241285 |
| | | | | WO2002102303 |
| | | | | WO2002102303 |
| | C. difficile | Bezlotoxumab | 4NP4 (Protein Data | |
| | Exotoxin B | | Bank) | |
| | (TcdB) | | DB13140 | |
| | | | (Drugbank) | |
| | B. anthracis | Obiltoxaximab | DB05336 | |
| | (Anthrax) | | (Drugbank) | |
| | protective | | | |
| | antigen 1 (PA1) | | | |
| | S. aureus | ASN-100 (Arsanis) | | |
| | cytotoxins | | | |
| Allergy & | IgE | Omalizumab | DB00042 | |
| Asthma | | | (Drugbank) | |
| | IL-5 | Mepolizumab | | |
| | IL-5 | Reslizumab | DB06602 | US20160102144C |
| | | | (Drugbank) | A2962944A1 |
| | | | | CN107073114 |
| | | | | EP3191513A1 |
| | | | | WO2016040007 |
| NASH | | | | |
| | CD3 | Muromonab- | DB00075 | |
| | | CD3 | (Drugbank) | |
| Cardiovascular | PCSK9 | Evelocumab | DB09303 | |
| | | | (Drugbank); D10557 | |
| | | | (KEGG Drug) | |
| | PCSK9 | Alirocumab | DB09302 | |
| Oncology | VEGF | Bevacizumab | DB00112 | |
| | CD19 | Blinatunomab | DB09052 | |
| | PDL-1 | | | |
| | matrix metallo- | Andecaliximab | | |
| | proteinase 9 | | | |
| | (MMP-9) | | | |
| Other/Misc | RANK Ligand | Denosumab | DB06643 | |
| | (RANKL) | | (Drugbank) | |
| | CD3/EpCAM | Catumaxomab | | |

The foregoing sequences are incorporated by reference herein in their entireties.

1. A method of producing recombinant secretory immunoglobulin A (sIgA) protein in a eukaryotic expression system, comprising:
co-transforming a eukaryotic cell with at least four different nucleic acid constructs comprising respective sequences encoding for each component polypeptide of said immunoglobulin, wherein at least one nucleic acid construct comprises a sequence encoding a light chain (LC), at least one nucleic acid construct comprises a sequence encoding a heavy chain (HC), at least one nucleic acid construct comprises a sequence encoding a joining chain (JC), and at least one nucleic acid construct comprises a sequence encoding a secretory component (SC) of said sIgA,
wherein each nucleic acid construct comprises the same promoter and same signal sequence, such that each of the immunoglobulin component polypeptides will be targeted to the same organelle of the cell for expression and assembly,
wherein said immunoglobulin component polypeptides are assembled in said eukaryotic cell to yield said recombinant sIgA protein.

2. The method of claim 1, wherein each promoter is derived from a protein storage gene that is operably linked to a DNA sequence that encodes for a protein storage-specific signal sequence capable of targeting a polypeptide linked thereto to a protein storage organelle of the eukaryotic cell.

3. The method of claim 2, wherein the protein storage gene and/or signal peptide are native to the eukaryotic cell.

4. The method of claim 2, wherein the protein storage gene and signal peptide are heterologous to the eukaryotic cell.

5. (canceled)

6. The method of claim 1, wherein said method has an expression yield of greater than 100 mg/kg of said secretory IgA.

7. The method of claim 1, wherein the transcriptional unit encoding for each sIgA component comprises codon optimized DNA sequences.

8. The method of claim 1, further comprising, transforming the eukaryotic cell with a nucleic acid construct comprising a selectable marker.

9. The method of claim 8, wherein said selectable marker is driven by the same promoter and signal peptide used for the immunoglobulin components.

10. The method of claim 1, wherein said eukaryotic cell is a plant cell, each of said constructs comprising a seed storage promoter protein, operably linked to a signal sequence capable of targeting a polypeptide linked thereto to a plant seed endosperm cell, said sequences encoding for each component polypeptide linked in translation frame with the signal sequence.

11. The method of claim 10, wherein the signal sequence encodes a rice glutelin signal sequence.

12. The method of claim 10, further comprising growing a plant from the transformed plant cell for a time sufficient to produce seeds containing the sIgA; and harvesting the seeds from the plant.

13.-14. (canceled)

15. The method of claim 1, wherein said eukaryotic cell is selected from the group consisting of wheat (Triticum sps.), rice (Oryza sps.), barley (Hordeum sps.), oats (Avena sps.), rye (Secale sps.), corn (maize) (Zea sps.), and millet (Pennisettum sps.), triticale, and sorghum.

16. (canceled)

17. A method of treating a condition in a human or non-human animal subject, comprising administering an effective amount of a recombinant secretory immunoglobulin A (sIgA) protein produced according to claim 1 to the subject in need thereof.

18. The method of claim 17, wherein the condition is selected from the group consisting of inflammatory conditions, infectious disease, cancer, auto-immune diseases, and combinations thereof.

19. The method of claim 17, wherein the condition is a skin condition or condition of the lung or nasal mucosa.

20. (canceled)

21. The method of claim 17, wherein the sIgA is administered orally, topically, or parenterally.

22. A transgenic cell produced according to the method of claim 1.

23. A transgenic seed produced according to the method of claim 1.

24.-25. (canceled)

26. The method of claim 1, wherein said recombinant sIgA protein is a fully assembled bioactive protein.

27. The method of claim 1, wherein said method has an expression yield of at least 1 g/kg of said secretory IgA.


SEQUENCE LISTING (29 sequences)

SEQ ID NO: 1 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 3 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 4 - protein, 121 aa, Homo sapiens [sequence omitted]

SEQ ID NO: 5 - protein, 107 aa, Homo sapiens [sequence omitted]

SEQ ID NO: 6 - protein, 107 aa, Homo sapiens [sequence omitted]

SEQ ID NO: 7 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 585 aa, Homo sapiens
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 9 - protein, 118 aa, Homo sapiens
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSS

SEQ ID NO: 10 - protein, 107 aa, Homo sapiens
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIK

SEQ ID NO: 11 - protein, 108 aa, Homo sapiens
DIQMTQSPSSLSASVGDRVTITCKASQDIRKYLNWYQQKPGKAPKLLIYYATSLADGVPSRFSGSGSGTDYTFTISSLQPEDIATYYCLQHGESPYTFGQGTKLEITR

SEQ ID NO: 12 - DNA, 1429 bp, Artificial Sequence
  Synthetic construct - codon optimized cDNA
gaggtgcagctcgtcgagagcgggggcggcttggtccagccgggccgctccctgaggctctcctgcgccgcctccggcttcaccttcgacgactacgcgatgcactgggtgcgccaggccccgggcaagggcctggagtgggtgtccgccatcacctggaactccggccacatcgactacgccgacagcgtggagggccgcttcaccatctcccgcgacaacgccaagaactccctgtacctccagatgaactccctcagggccgaggacaccgccgtgtactactgcgccaaggtgagctacctctccaccgcctcctccctcgactactgggggcagggcaccctcgtgaccgtgtcctccgcctccccgacctccccgaaggtgttcccgttgtccctgtgctccacccagccggacggcaacgtggtgatcgcctgcctggtccaaggcttcttcccgcaggagccgctctcggtcacctggtccgaatcgggccagggcgtgaccgcccgcaacttcccgccaagccaggacgcgagcggcgatctctacaccacctcgtcccagctcaccctcccggccacgcagtgcctcgccggcaagtccgtgacctgccacgtcaagcactacaccaacccgtcccaggacgtcaccgtgccgtgcccggtcccgagcaccccgccgaccccgagcccgtccaccccgccaaccccgtccccgagctgctgccacccgcgcctctccctgcacaggccggccttggaggacctcctcctcggctccgaggccaacctcacgtgcaccctcaccggcctccgcgacgcgtccggcgtcaccttcacgtggaccccgtcctccggcaagagcgccgtgcagggcccgccggagcgcgatctctgcggctgctactccgtctcctccgtgctcccgggctgcgccgagccgtggaaccacggcaagaccttcacctgcaccgccgcctacccggagagcaagaccccgctcaccgccaccctctccaagagcggcaacaccttccgcccggaggtccacctcctcccgccgccgtccgaggagctcgccctcaacgagctggtcaccctgacctgcctcgcccgcggcttcagcccgaaggacgtgctcgtgcgctggctccagggctcccaggagctcccgcgcgagaagtacctcacctgggcctcccgccaggagccatcccagggcaccaccaccttcgccgtgacctccatcctccgcgtggccgccgaggactggaagaagggcgacaccttctcctgcatggtcggccacgaggccctcccgctcgccttcacccagaagaccatcgaccgcctggccggcaagccgacccacgtgaacgtctccgtggtgatggccgaggtggacggcacctgctactgatagc

SEQ ID NO: 13 - DNA, 649 bp, Artificial Sequence
  Synthetic construct - codon optimized cDNA
gacatccagatgacccagtccccgtccagcctgagcgccagcgtcggcgacagggtgaccatcacctgcagggcctcccagggcatccgcaactacctcgcgtggtaccagcagaagccgggcaaggccccgaagctcctcatctacgccgcgtccaccctgcagtccggcgtgccgtcccgcttcagcggctccggcagcggcaccgacttcaccctcaccatctccagcctccagccggaggacgtcgccacctactactgccagcgctacaaccgcgccccgtacaccttcggccagggcaccaaggtcgagatcaagcgcaccgtggccgccccgtccgtgttcatcttcccgccgtccgacgagcagctgaagtccggcaccgcctccgtcgtgtgcctgctcaacaacttctacccgcgcgaggcgaaggtgcagtggaaggtggacaacgccctccagtccggcaactcccaggagagcgtgaccgagcaggacagcaaggacagcacctacagcctgtccagcaccctgaccctcagcaaggcggactacgagaagcacaaggtctacgcgtgcgaggtgacccaccagggcctctccagcccggtgaccaagtccttcaaccgcggcgagtgctgatagc

SEQ ID NO: 14 - DNA, 418 bp, Artificial Sequence
  Synthetic construct - codon optimized cDNA
caggaggacgagcgcatcgtgctggtggacaacaagtgcaagtgcgcccgcatcacctccaggatcatccgcagcagcgaggacccgaacgaggacatcgtcgagaggaacatccgcatcatcgtcccgctgaacaaccgcgagaacatcagcgacccgaccagcccgctgcgcacccgcttcgtgtaccacctgtccgacctctgcaagaagtgcgacccaaccgaggtcgagctggacaaccagatcgtgaccgccacccagtccaacatctgcgacgaggacagcgccaccgagacctgctacacctacgacaggaacaagtgctacaccgcggtcgtgccgctcgtgtacggcggcgagaccaagatggtcgagaccgccctgaccccggacgcgtgctacccggactgatagc

SEQ ID NO: 15 - DNA, 1762 bp, Artificial Sequence
  Synthetic construct - codon optimized cDNA
aagtccccgatcttcggccccgaggaggtgaactccgtcgagggcaacagcgtgagcatcacctgctactacccgcccacctccgtgaaccgccacaccaggaagtactggtgtcgccagggcgcgcgcggcggctgcatcaccctcatcagcagcgagggctacgtgtcctccaagtacgccggccgcgccaacctcaccaacttcccggagaacggcacgttcgtggtgaacatcgcccagctctcccaggacgacagcggccggtacaagtgcggcctcggcatcaacagccgcggcctctccttcgacgtctccctggaggtgtcccaggggccgggcctgctcaacgacaccaaggtgtacacggtggacctcggccgcaccgtcaccatcaactgcccgttcaagaccgagaacgcgcagaagcgcaagagcctgtacaagcagatcggcctctacccggtgctcgtgatcgacagcagcggctacgtcaacccgaactacaccggccgcatcaggctggacatccagggcaccggccagctgctgttctccgtcgtcatcaaccagctgcgcctctccgacgcgggccagtacctgtgccaggccggcgacgacagcaactccaacaagaagaacgccgacctccaggtgctcaagccggagccggagctcgtctacgaggacctccgcggctccgtgaccttccactgcgccctgggcccggaggtcgccaacgtggccaagttcctctgcaggcagagcagcggcgagaattgcgacgtcgtggtcaataccctgggcaagcgcgccccggcgttcgagggcaggatcctcctgaacccgcaggacaaggacggctccttctccgtggtcatcaccggcctgcgcaaggaggacgccggcaggtacctctgcggcgcccacagcgacggccagctccaggagggcagcccgatccaggcgtggcagctcttcgtgaacgaggagagcaccatcccgcgctccccgaccgtcgtcaagggcgtggccggcggcagcgtggccgtgctctgcccgtacaaccgcaaggagagcaagtccatcaagtattggtgtctctgggagggcgcccagaacggccgctgcccgctgctcgtggacagcgaggggtgggtgaaggcccagtacgagggccgcctcagcctgctggaggagccgggcaacggcaccttcaccgtcatcctcaatcagctcaccagccgcgacgccggcttctattggtgcctgaccaacggcgacaccctctggcgcaccaccgtggagatcaagatcatcgagggcgagccgaacctcaaggtgccggggaacgtcaccgcggtcctcggggagaccctgaaggtcccgtgccacttcccgtgcaagttctcctcctacgagaaatactggtgcaagtggaacaacaccggctgccaagccctcccgtcccaggatgagggcccgtccaaggccttcgtcaactgcgacgagaactcccgcctcgtcagcctgaccctcaacctggtgacccgcgcggacgagggctggtactggtgcggcgtgaagcagggccacttctacggcgagaccgccgccgtgtacgtcgccgtggaggagcgcaaggccgcgggctcccgcgacgtgtccctcgccaaggccgacgccgcgccggacgagaaggtgctggacagcggcttccgcgagatcgagaacaaggccatccaggacccgaggtgatagc

SEQ ID NO: 16 - DNA, 785 bp, Oryza sativa
tcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagagagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcggccgcttcattagtcctacaacaacatggcatccataaatcgccccatagttttcttcacagtttgcttgttcctcttgtgcgatggctccctagcc

SEQ ID NO: 17 - DNA, 285 bp, Agrobacterium tumefaciens
tcgagtctagacgcgtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 18 - protein, 499 aa, Oryza sativa
MASINRPIVFFTVCLFLLCDGSLAQQLLGQSTSQWQSSRRGSPRGCRFDRLQAFEPIRSVRSQAGTTEFFDVSNELFQCTGVSVVRRVIEPRGLLLPHYTNGASLVYIIQGRGITGPTFPGCPETYQQQFQQSGQAQLTESQSQSHKFKDEHQKIHRFRQGDVIALPAGVAHWCYNDGEVPVVAIYVTDINNGANQLDPRQRDFLLAGNKRNPQAYRREVEEWSQNIFSGFSTELLSEAFGISNQVARQLQCQNDQRGEIVRVERGLSLLQPYASLQEQEQGQMQSREHYQEGGYQQSQYGSGCPNGLDETFCTMRVRQNIDNPNRADTYNPRAGRVTNLNSQNFPILNLVQMSAVKVNLYQNALLSPFWNINAHSIVYITQGRAQVQVVNNNGKTVFNGELRRGQLLIVPQHYVVVKKAQREGCAYIAFKTNPNSMVSHIAGKSSIFRALPTDVLANAYRISREEAQRLKHNRGDEFGAFTPLQYKSYQDVYNVAESS

SEQ ID NO: 19 - DNA, 2499 bp, Artificial Sequence
  Synthetic expression construct
tcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagagagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcggccgcttcattagtcctacaacaacatggcatccataaatcgccccatagttttcttcacagtttgcttgttcctcttgtgcgatggctccctagccgaggtgcagctcgtcgagagcgggggcggcttggtccagccgggccgctccctgaggctctcctgcgccgcctccggcttcaccttcgacgactacgcgatgcactgggtgcgccaggccccgggcaagggcctggagtgggtgtccgccatcacctggaactccggccacatcgactacgccgacagcgtggagggccgcttcaccatctcccgcgacaacgccaagaactccctgtacctccagatgaactccctcagggccgaggacaccgccgtgtactactgcgccaaggtgagctacctctccaccgcctcctccctcgactactgggggcagggcaccctcgtgaccgtgtcctccgcctccccgacctccccgaaggtgttcccgttgtccctgtgctccacccagccggacggcaacgtggtgatcgcctgcctggtccaaggcttcttcccgcaggagccgctctcggtcacctggtccgaatcgggccagggcgtgaccgcccgcaacttcccgccaagccaggacgcgagcggcgatctctacaccacctcgtcccagctcaccctcccggccacgcagtgcctcgccggcaagtccgtgacctgccacgtcaagcactacaccaacccgtcccaggacgtcaccgtgccgtgcccggtcccgagcaccccgccgaccccgagcccgtccaccccgccaaccccgtccccgagctgctgccacccgcgcctctccctgcacaggccggccttggaggacctcctcctcggctccgaggccaacctcacgtgcaccctcaccggcctccgcgacgcgtccggcgtcaccttcacgtggaccccgtcctccggcaagagcgccgtgcagggcccgccggagcgcgatctctgcggctgctactccgtctcctccgtgctcccgggctgcgccgagccgtggaaccacggcaagaccttcacctgcaccgccgcctacccggagagcaagaccccgctcaccgccaccctctccaagagcggcaacaccttccgcccggaggtccacctcctcccgccgccgtccgaggagctcgccctcaacgagctggtcaccctgacctgcctcgcccgcggcttcagcccgaaggacgtgctcgtgcgctggctccagggctcccaggagctcccgcgcgagaagtacctcacctgggcctcccgccaggagccatcccagggcaccaccaccttcgccgtgacctccatcctccgcgtggccgccgaggactggaagaagggcgacaccttctcctgcatggtcggccacgaggccctcccgctcgccttcacccagaagaccatcgaccgcctggccggcaagccgacccacgtgaacgtctccgtggtgatggccgaggtggacggcacctgctactgatagctcgagtctagacgcgtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 20 - DNA, 1719 bp, Artificial Sequence
  Synthetic expression construct
tcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagagagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcggccgcttcattagtcctacaacaacatggcatccataaatcgccccatagttttcttcacagtttgcttgttcctcttgtgcgatggctccctagccgacatccagatgacccagtccccgtccagcctgagcgccagcgtcggcgacagggtgaccatcacctgcagggcctcccagggcatccgcaactacctcgcgtggtaccagcagaagccgggcaaggccccgaagctcctcatctacgccgcgtccaccctgcagtccggcgtgccgtcccgcttcagcggctccggcagcggcaccgacttcaccctcaccatctccagcctccagccggaggacgtcgccacctactactgccagcgctacaaccgcgccccgtacaccttcggccagggcaccaaggtcgagatcaagcgcaccgtggccgccccgtccgtgttcatcttcccgccgtccgacgagcagctgaagtccggcaccgcctccgtcgtgtgcctgctcaacaacttctacccgcgcgaggcgaaggtgcagtggaaggtggacaacgccctccagtccggcaactcccaggagagcgtgaccgagcaggacagcaaggacagcacctacagcctgtccagcaccctgaccctcagcaaggcggactacgagaagcacaaggtctacgcgtgcgaggtgacccaccagggcctctccagcccggtgaccaagtccttcaaccgcggcgagtgctgatagctcgagtctagacgcgtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 21 - DNA, 1488 bp, Artificial Sequence
  Synthetic expression construct
tcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagagagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcggccgcttcattagtcctacaacaacatggcatccataaatcgccccatagttttcttcacagtttgcttgttcctcttgtgcgatggctccctagcccaggaggacgagcgcatcgtgctggtggacaacaagtgcaagtgcgcccgcatcacctccaggatcatccgcagcagcgaggacccgaacgaggacatcgtcgagaggaacatccgcatcatcgtcccgctgaacaaccgcgagaacatcagcgacccgaccagcccgctgcgcacccgcttcgtgtaccacctgtccgacctctgcaagaagtgcgacccaaccgaggtcgagctggacaaccagatcgtgaccgccacccagtccaacatctgcgacgaggacagcgccaccgagacctgctacacctacgacaggaacaagtgctacaccgcggtcgtgccgctcgtgtacggcggcgagaccaagatggtcgagaccgccctgaccccggacgcgtgctacccggactgatagctcgagtctagacgcgtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 22 - DNA, 2832 bp, Artificial Sequence
  Synthetic expression construct
tcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagagagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcggccgcttcattagtcctacaacaacatggcatccataaatcgccccatagttttcttcacagtttgcttgttcctcttgtgcgatggctccctagccaagtccccgatcttcggccccgaggaggtgaactccgtcgagggcaacagcgtgagcatcacctgctactacccgcccacctccgtgaaccgccacaccaggaagtactggtgtcgccagggcgcgcgcggcggctgcatcaccctcatcagcagcgagggctacgtgtcctccaagtacgccggccgcgccaacctcaccaacttcccggagaacggcacgttcgtggtgaacatcgcccagctctcccaggacgacagcggccggtacaagtgcggcctcggcatcaacagccgcggcctctccttcgacgtctccctggaggtgtcccaggggccgggcctgctcaacgacaccaaggtgtacacggtggacctcggccgcaccgtcaccatcaactgcccgttcaagaccgagaacgcgcagaagcgcaagagcctgtacaagcagatcggcctctacccggtgctcgtgatcgacagcagcggctacgtcaacccgaactacaccggccgcatcaggctggacatccagggcaccggccagctgctgttctccgtcgtcatcaaccagctgcgcctctccgacgcgggccagtacctgtgccaggccggcgacgacagcaactccaacaagaagaacgccgacctccaggtgctcaagccggagccggagctcgtctacgaggacctccgcggctccgtgaccttccactgcgccctgggcccggaggtcgccaacgtggccaagttcctctgcaggcagagcagcggcgagaattgcgacgtcgtggtcaataccctgggcaagcgcgccccggcgttcgagggcaggatcctcctgaacccgcaggacaaggacggctccttctccgtggtcatcaccggcctgcgcaaggaggacgccggcaggtacctctgcggcgcccacagcgacggccagctccaggagggcagcccgatccaggcgtggcagctcttcgtgaacgaggagagcaccatcccgcgctccccgaccgtcgtcaagggcgtggccggcggcagcgtggccgtgctctgcccgtacaaccgcaaggagagcaagtccatcaagtattggtgtctctgggagggcgcccagaacggccgctgcccgctgctcgtggacagcgaggggtgggtgaaggcccagtacgagggccgcctcagcctgctggaggagccgggcaacggcaccttcaccgtcatcctcaatcagctcaccagccgcgacgccggcttctattggtgcctgaccaacggcgacaccctctggcgcaccaccgtggagatcaagatcatcgagggcgagccgaacctcaaggtgccggggaacgtcaccgcggtcctcggggagaccctgaaggtcccgtgccacttcccgtgcaagttctcctcctacgagaaatactggtgcaagtggaacaacaccggctgccaagccctcccgtcccaggatgagggcccgtccaaggccttcgtcaactgcgacgagaactcccgcctcgtcagcctgaccctcaacctggtgacccgcgcggacgagggctggtactggtgcggcgtgaagcagggccacttctacggcgagaccgccgccgtgtacgtcgccgtggaggagcgcaaggccgcgggctcccgcgacgtgtccctcgccaaggccgacgccgcgccggacgagaaggtgctggacagcggcttccgcgagatcgagaacaaggccatccaggacccgaggtgatagctcgagtctagacgcgtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 23 - DNA, 60 bp, Triticum aestivum
atggctaagcgcctggtcctctttgcggcagtagtcgtcgccctcgtggctctcaccgcc

SEQ ID NO: 24 - DNA, 72 bp, Oryza sativa
atggcaactaccattttctctcgtttttctatatacttttgtgctatgctattatgccagggttctatggcc

SEQ ID NO: 25 - DNA, 85 bp, Oryza sativa
atgtggacattaacaaactctatcttaacatctagtcgatcactactttacttcactataaaaggaccaacatatatcatccatt

SEQ ID NO: 26 - DNA, 72 bp, Oryza sativa
atggcgagttccgttttctctcggttttctatatacttttgtgttcttctattatgccatggttctatggcc

SEQ ID NO: 27 - DNA, 69 bp, Oryza sativa
atgaagatcattttcgtatttgctctccttgctattgttgcatgcaacgcttctgcacggtttgatgct

SEQ ID NO: 28 - DNA, 63 bp, Oryza sativa
atggccgcccgcgccgccgccgccgcgttcctgctgctgctcatcgtcgttggtcaccgcgcc

SEQ ID NO: 29 - DNA, 63 bp, Hordeum vulgare
atggctaagcggctggtcctctttgtggcggtaatcgtcgccctcgtggctctcaccaccgcc